(19) United States
(12) Patent Application Publication
(10) Pub. No.: US 2009/0038024 A1
(43) Pub. Date: Feb. 5, 2009

(54) CAP/SORBS1 AND DIABETES

(75) Inventors: Ping H. Wang, Irvine, CA (US); Lee-Ming Chuang, Taipei Hsien (TW); Arthur Lander, Laguna Beach, CA (US)

Correspondence Address: Weaver Austin Villeneuve & Sampson LLP, P.O. BOX 70250, OAKLAND, CA, 94612-0250, US

(73) Assignees: THE REGENTS OF THE UNIVERSITY OF CALIFORNIA, Oakland, CA (US); NATIONAL TAIWAN UNIVERSITY, Taipei (TW)

(21) Appl. No.: 12/141,782

(22) Filed: Jun. 18, 2008

Related U.S. Application Data
(60) Provisional application No. 60/945,062, filed on Jun. 19, 2007.

(51) Int. Cl. A01K 67/027; G01N 33/566; G01N 33/53; A61K 31/7088; A61P 3/10

(52) U.S. Cl. 800/18; 436/501; 435/7.1; 514/44

(57) ABSTRACT

The present invention provides methods, compositions, and kits useful for modulating insulin/glucose homeostasis in a subject by modulating CAP/SORBS1. In addition, the invention provides a variety of prescreening and screening methods aimed at identifying agents that modulate insulin/glucose homeostasis. Methods of the invention can involve assaying test agent binding to CAP/SORBS1 polypeptides or polynucleotides. Alternatively, test agents can be screened for their ability to alter the level of CAP/SORBS1 polypeptides, polynucleotides, or action.

### CROSS-REFERENCE TO RELATED APPLICATIONS

This application claims benefit of and priority to U.S. Ser. No. 60/945,062, filed on Jun. 19, 2007, which is incorporated herein by reference in its entirety for all purposes.

### FIELD OF THE INVENTION

This invention pertains to methods and compositions relating to the role of CAP/SORBS1 in modulating insulin/glucose homeostasis.

### BACKGROUND OF THE INVENTION

Insulin resistance and 13 cell dysfunction are two critical factors during the development of Type 2 diabetes. A mechanistic link between insulin resistance and 13 cell dysfunction in the majority of human diabetes is not yet clear, but there is substantial evidence that a defect in receptor signaling may link insulin resistance to islet cell dysfunction (Saltiel and Kahn; Kulkami et al; Nandi et al; Plum et al). Insulin regulates glucose homeostasis by activating insulin receptor signaling pathways to control glucose metabolism (Saltiel and Kahn). Glucose disposal is in part mediated by the glucose transporter GLUT4 in muscle and adipose tissues. Insulin stimulates the translocation of Glut4 from intracellular space to the plasma membrane to facilitate glucose uptake into the cells (James et al; Saltiel and Kahn; Minokoshi et al). The insulin signaling pathways required for Glut4 translocation have been intensely studied in the last two decades, but the mechanisms of regulation remain incompletely understood (Zhou et al). In addition to glucose transport, insulin regulation of glycogen synthesis also plays a role in glucose homeostasis (DeFronzo). Three pathways of insulin signaling may regulate glucose homeostasis. The most defined pathway is mediated through IRS, PI3 kinase, and Akt; this pathway is clearly required to maintain physiological glucose homeostasis (Saltiel and Kahn; Zhou et al). The second pathway involves CAP, APS, CrkII, and TC10 (Saltiel and Kahn), and the third pathway involves phospholipase C and IP3 (Zhou et al). Accumulating evidence has suggested that the phospholipase C/IP3 pathway does not play a critical role in the regulation insulin-mediated glucose disposal.

### SUMMARY OF THE INVENTION

The invention provides a non-human knockout mammal that has a disruption in an endogenous CAP/SORBS1 gene. The disruption results in the mammal exhibiting a decreased level of CAP/SORBS1 as compared to a wild-type mammal. The mammal can be, for example, a rodent, an equine, a bovine, a porcine, a lagomorph, a feline, a canine, a murine, a caprine, an ovine, and a non-human primate. Exemplary disruptions include an insertion, a deletion, a frameshift mutation, a substitution, and a stop codon. In certain embodiments, the disruption includes an insertion of an expression cassette into the endogenous CAP/SORBS1 gene. If desired, the expression cassette can include a selectable marker, e.g., a fusion of β-galactosidase and neomycin phosphotransferase II. The disruption can be in a somatic cell or a germ cell. The knockout mammal can be homozygous or heterozygous for the disrupted CAP/SORBS gene.

Another aspect of the invention relates to prescreening and screening methods. In one embodiment, a method of prescreening for an agent useful in the prophylaxis or treatment of a disorder of insulin homeostasis and/or glucose homeostasis entails:

(a) contacting a test agent with a CAP/SORBS1 polypeptide;

(b) determining whether the test agent specifically binds to the polypeptide; and

(c) if the test agent specifically binds to the polypeptide, selecting the test agent as potentially useful in the prophylaxis or treatment of a disorder of insulin homeostasis and/or glucose homeostasis.

In another embodiment, a method of prescreening for an agent useful in the prophylaxis or treatment of a disorder of insulin homeostasis and/or glucose homeostasis entails:

(a) contacting a test agent with a CAPS/SORBS1 polynucleotide;

(b) determining whether the test agent specifically binds to the polynucleotide; and

(c) if the test agent specifically binds to the polynucleotide, selecting the test agent as potentially useful in the prophylaxis or treatment of a disorder of insulin homeostasis and/or glucose homeostasis. In a variation of this embodiment, the CAP/SORBS1 polynucleotide includes the CAP/SORBS1 promoter.

In one embodiment, a method of screening for an agent useful in the prophylaxis or treatment of a disorder of insulin homeostasis and/or glucose homeostasis entails:

(a) contacting a test agent with a cell that expresses CAP/SORBS1 in the absence of test agent, or with a fraction of said cell;

(b) determining whether the test agent modulates CAP/SORBS1 expression and/or activity; and

(c) if the test agent modulates CAP/SORBS1 expression and/or activity, selecting the test agent as potentially useful in the prophylaxis or treatment of a disorder of insulin homeostasis and/or glucose homeostasis. This screening method can, optionally, entail the step of combining the selected test agent with a pharmaceutically acceptable carrier. In a variation of this embodiment, the ability of the selected test agent to prevent or treat a disorder of insulin homeostasis and/or glucose homeostasis can be measured in an animal model.

In any of the above-described prescreening or screening methods of the invention, the test agent can conveniently be contacted with the other assay component(s) in vitro. Any of these methods can additionally include the step of recording any selected test agent in a database of agents that are potentially useful in the prophylaxis or treatment of a disorder of insulin homeostasis and/or glucose homeostasis.

The invention also provides a method of in vivo screening for an agent useful in the prophylaxis or treatment of a disorder of insulin homeostasis and/or glucose homeostasis. This method entails:

(a) selecting a modulator of CAP/SORBS1 expression or activity as a test agent; and

(b) measuring the ability of the selected test agent to prevent or treat a disorder of insulin homeostasis and/or glucose homeostasis in an animal model. In particular embodiments, the modulator includes an enhancer of CAP/SORBS1 expression or activity.

In an alternative embodiment, a method of in vivo screening for an agent useful in the prophylaxis or treatment of a disorder of insulin homeostasis and/or glucose homeostasis entails:

(a) administering a test agent to a mammal exhibiting an abnormal level of CAP/SORBS1; and

(b) determining glucose tolerance and/or insulin resistance and/or insulin sensitivity;

wherein a test agent that modulates glucose tolerance and/or modulates insulin resistance is selected as a candidate agent useful in the prophylaxis or treatment of a disorder of insulin homeostasis and/or glucose homeostasis. In certain embodiments, the abnormal level of CAP/SORBS1 is a reduced level as compared to a normal level. In particular embodiments, the mammal is a non-human mammal. For example, the mammal can be a knockout mammal that includes a disruption in an endogenous CAP/SORBS1 gene, wherein the disruption results in the mammal exhibiting a reduced level of CAP/SORBS1 as compared to a wild-type mammal. For any of the above-described screening methods, the disorder of insulin homeostasis and/or glucose homeostasis can be, for example, metabolic syndrome, pre-diabetes, diabetes, and/or obesity.

The invention also provides a method of determining whether a subject is a candidate for CAP/SORBS1-based therapy. The method entails measuring the level and/or activity of CAP/SORBS1 in a biological sample from the subject, wherein an altered level or activity of CAP/SORBS1, relative to a normal level, indicates that the subject is a candidate for CAP/SORBS1-based therapy. In particular embodiments, the altered level is a reduced level, relative to a normal level. In certain embodiments, the subject has at least one symptom of a disorder of insulin homeostasis and/or glucose homeostasis. The disorder of insulin homeostasis and/or glucose homeostasis can include, for example, metabolic syndrome, pre-diabetes, diabetes, and/or obesity. In exemplary embodiments, the CAP/SORBS 1-based therapy includes treatment with an agonist of peroxisome proliferator activated receptor gamma (PPARγ).

Another aspect of the invention is a method of prophylaxis or treatment of a disorder of insulin homeostasis and/or glucose homeostasis. This method entails administering an agent that modulates CAP/SORBS1 activity to a subject at risk for, or having, a disorder of insulin homeostasis and/or glucose homeostasis, provided that said level of CAP/SORBS1 activity is increased by means other than treatment with an agonist of peroxisome proliferator activated receptor gamma (PPARγ). In particular embodiments, the modulator enhances CAP/SORBS1 activity. The disorder of insulin homeostasis and/or glucose homeostasis can include, for example, metabolic syndrome, pre-diabetes, diabetes, and/or obesity. In exemplary embodiments, the agent is a CAP/SORBS1 polypeptide or polynucleotide. Variations of this method can additionally include determining glucose tolerance, and/or insulin resistance, and/or insulin sensitivity, and/or metabolic syndrome in the subject after treatment to modulate CAP/SORBS1 activity to determine whether the subject is responding to said treatment.

### BRIEF DESCRIPTION OF THE DRAWINGS

FIG. 1. Insertional mutations in CAP. Numbered boxes represent exons. The mutated allele yielded an in-frame fusion transcript with exons 1-4 and P3-Geo in the RRD158 clone and with exons 1-17 and P3-Geo in the RRP094 clone.

FIG. 2A-D. Genotyping of mice derived from RRD158. Tail genomic DNA was used for genotyping of mice derived from RRD158 and RRP094 ES cell line. A. Primer sets used for PCR screening. B: Screening of D158a mice carrying the mutated CAP gene. PCR was carried out with a pair of primer sets A+B and A+C. The presence of 1047 bp and 964 bp fragments indicated the mutated allele with the trapped β-Geo. Amplification of a fragment of GAPDH served as control. C: Screening of D158a heterozygous CAP^{(+/-)} and homozygous CAP^{(-/-)} mice. PCR was carried out with primer sets D+E and D+F. PCR products yielded a 572 bp fragment (mutated allele) and a 644 bp fragment (wildtype CAP allele). D: Screening of P094a mice carrying the mutated CAP gene. PCR was performed with primer sets G+H and G+I. The presence of 1042 bp and 931 bp fragments indicated the mutated allele with the trapped β-Geo. DNA isolated from RRP094 ES cells served as positive control. M10.1 carries wildtype CAP, M10.2 carries mutated allele.

FIG. 3. Expression of Wildtype CAP gene in various mouse tissues. RNA was extracted from tissues isolated from wildtype (+/+), heterozygous CAP knockout (+/−), and homozygous CAP knockout (−/−) mice. Wildtype CAP mRNA was assessed by RT-PCR. Equal amounts of RNAs from each tissue were used for RT. Two different primers sets were used for PCR, a pair of primers franking CAP exon 1 to exon 6 were used in the experiment on the left panel, and another pair of primers franking exon 2 to exon 5 were used in the experiment on right panel (skeletal muscle and liver). GAPDH served as controls.

FIG. 4A-C. Expression of CAP protein in the knockout mice and in vivo activation of insulin receptor. A. CAP protein detected by western blots. Tissues were isolated from the wildtype and homozygous D158a CAP^{(-/-)} mice and separated with 8% SDS-PAGE and immunoblotted with anti-CAP antibodies or anti-actin antibodies. B. CAP protein in P094a mice. C. In vivo activation of myocardial insulin receptor. The mice were injected with insulin or vehicles for 2 min via inferior vena cava and the myocardium was homogenated, immunoprecipitated with anti-insulin receptor antibodies, and then immunoblotted with anti-phosphotyrosine antibodies.

FIG. 5A-D. Glucose Tolerance Test (GTT) and Insulin Tolerance Test (ITT). Glucose tolerance test (2 g/Kg B.W. D-Glucose, i.p.) was performed in 24-weeks-old CAP^{(-/-)} and CAP^{(+/+)} mice, the littermate CAP^{(+/+)} mice served as controls. These mice derived from four independent litters. Tail blood was sampled and analyzed with an automatic glucose analyzer. A: GTT of non-pregnant female mice: D158a mice were used in this series of study. The graph represents blood glucose data from female mice (n=10 in each group), data were presented as mean±SEM. *p<0.05. B: GTT of male mice. The graph represents the glucose levels during GTT in male D158a mice (control, heterozygous, and homozygous knockout, n=9). *p<0.05, CAP^{(-/-)} vs. CAP^{(+/+)}. C: ITT in non-pregnant female mice. After overnight fasting, mice were injected with insulin (0.75 U/Kg BW). The data (mean±SEM) represent the blood glucose levels from 0 to 90 min during ITT in female mice. N=10 in each group, p<0.05. D: Plasma insulin levels in response to D-glucose. Plasma insulin levels were analyzed by ELISA (Crystal Chem). The data represent mean±SD from 8 littermate controls and 9 knockout female mice. Insulin levels after D-Glucose injection increased in the knockout mice, which suggested hyperinsulinemia secondary to insulin resistance in the CAP^{(-/-)} female mice. *p<0.05, CAP^{(-/-)} vs. CAP^{(+/+)}.

FIG. 6A-B. Body weights were not changed in the CAP KO mice. The body weights of the D158a mice used for preliminary study. Body weight data derived from 9-24 mice in each group at various time points. A: Males; B: Females.

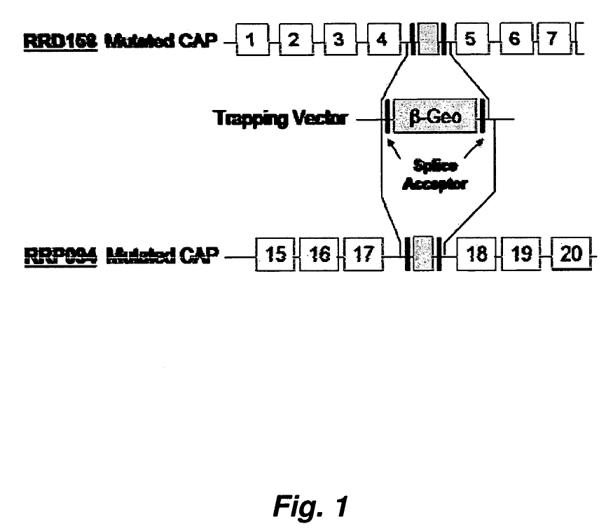

### DETAILED DESCRIPTION

### I. Introduction

CAP was first discovered in 1998 (Ribon V et al). Cbl is recruited to the insulin receptor by interacting with CAP, through one of three adjacent SH3 domains in the carboxy terminus of CAP. Upon insulin stimulation, the CAP-Cbl complex moves to a caveolin-enriched membrane fraction (Ribon V et al). Since overexpression of a dominant negative CAP in 3T3-L1 adipocytes blocked insulin-stimulated glucose uptake, it has been proposed that insulin-mediated glucose transport in fat and muscle may involve CAP (Liu J et al; Baumann et al). A different laboratory published additional evidence corroborating the involvement of CAP/Cbl/Crk during insulin activation of Glut4 (Standaert et al.). However, using a knock-down strategy, two other laboratories showed that insulin signaling to glucose transport did not require the CAP/TC10 pathway (Mitra et al.; Jebailey et al.). These conflicting results, all based on in vitro models, were unable to conclude whether CAP is involved in the regulation of glucose homeostasis in vivo.

Cbl was initially proposed as an adapter protein interacting with CAP and is part of the insulin signaling network to promote glucose transport. But the Cbl knockout mice exhibited a surprising phenotype. Cbl knockout mice showed increased insulin sensitivity and decreased adiposity, through activation of AMPK pathways independent of insulin signaling (Molero et al.). Cbl knockout mice also showed increased food uptake and increased fatty acid oxidation (Molero et al.). This raised an alternative possibility that CAP may interact with Cbl, modulate AMPK, and thus regulate glucose disposal through mechanism independent of insulin receptor signaling pathways. Moreover, earlier evidence showed that the CAP/TC10 pathway was independent of PI3 kinase (Baumann et al.). But recent studies suggested that Cbl may interact with the p85 subunit of PI3 kinase and modulate glycogen synthesis (Katsanakis et al; Standaert et al).

The human homolog for mouse CAP is SORBS1 (SH3-domain-containing 1) (Lin W et al). CAP/SORBS1 is among a number of genes whose expression is up-regulated by thiazolidinediones, a class of anti-diabetes agents that bind to PPARγ and reduce insulin resistance (Bogacka et al; Ribon et al; Lin W et al.). In humans, the A-allele of the T228A polymorphism in exon 7 of SORBS1 gene has a protective role against Type 2 diabetes (Relative Risk: 0.668, 95% CI 0.265-0.821) (Lin W et al). However, the previous in vitro studies left the physiological role of CAP/SORBS1 uncertain.

The work described herein demonstrates that in vivo disruption of CAP/SORBS1 leads to insulin resistance and glucose intolerance in vivo. CAP/SORBS1 knockout animals have been produced which are useful for studying the role of CAP/SORBS1 in insulin/glucose homeostasis and related disorders. The in vivo demonstration that reduction in CAP/SORBS1 produces insulin resistance and glucose intolerance provides a basis for pre-screening and screening for agents that modulate insulin/glucose homeostasis using CAP/SORBS1 as a target. This in vivo demonstration also provides a basis for a treatment method based on modulating (e.g., enhancing) CAP/SORBS1 expression or activity, as well as a diagnostic method based on screening for altered (e.g., reduced) CAP/SORBS1 as an indicator of whether a subject is a candidate for CAP/SORBS 1-based therapy.

### II. Definitions

Terms used in the claims and specification are defined as set forth below unless otherwise specified.

The following terms encompass polypeptides that are identified in Genbank by the following designations, as well as polypeptides that are at least about 70% identical to polypeptides identified in GenBank by these designations: c-Cbl-associated protein (CAP), Ponsin, SH3P12, SORBS1. In alternative embodiments, these terms encompass polypeptides identified in GenBank by these designations and sharing at least about 80, 90, 95, 96, 97, 98, or 99% identity.

A “modulator” of a polypeptide is either an inhibitor or an enhancer of an action or function of the polypeptide.

A “non-selective” modulator of a polypeptide is an agent that modulates other members of the same family of polypeptides at the concentrations typically employed for modulation of the particular polypeptide.

A “selective” modulator of a polypeptide significantly modulates the particular polypeptide at a concentration at which other members of the same family of polypeptides are not significantly modulated.

A modulator “acts directly on” a polypeptide when the modulator exerts its action by interacting directly with the polypeptide.

A modulator “acts indirectly on” a polypeptide when the modulator exerts its action by interacting with a molecule other than the polypeptide, which interaction results in modulation of an action or function of the polypeptide.

An “inhibitor” or “antagonist” of a polypeptide is an agent that reduces, by any mechanism, any action or function of the polypeptide, as compared to that observed in the absence (or presence of a smaller amount) of the agent. An inhibitor of a polypeptide can affect: (1) the expression, mRNA stability, protein trafficking, modification (e.g., phosphorylation), or degradation of a polypeptide, or (2) one or more of the normal action or functions of the polypeptide. An inhibitor of a polypeptide can be non-selective or selective. Preferred inhibitors (antagonists) are generally small molecules that act directly on, and are selective for, the target polypeptide.

An “enhancer” or “activator” is an agent that increases, by any mechanism, any polypeptide action or function, as compared to that observed in the absence (or presence of a smaller amount) of the agent. An enhancer of a polypeptide can affect: (1) the expression, mRNA stability, protein trafficking, modification (e.g., phosphorylation), or degradation of a polypeptide, or (2) one or more of the normal actions or functions of the polypeptide. An enhancer of a polypeptide can be non-selective or selective. Preferred enhancers (activators) are generally small molecules that act directly on, and are selective for, the target polypeptide.

The terms “polypeptide” and “protein” are used interchangeably herein to refer a polymer of amino acids, and unless otherwise limited, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Exemplary atypical amino acids, include, for example, those described in International Publication No. WO 90/01940 as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3-(and 4-) hydroxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “specific binding” is defined herein as the preferential binding of binding partners to one another (e.g., two polypeptides, a polypeptide and nucleic acid molecule, or two nucleic acid molecules) at specific sites. The term “specifically binds” indicates that the binding preference (e.g., affinity) for the target molecule/sequence is at least 2-fold, more preferably at least 5-fold, and most preferably at least 10- or 20-fold over a non-specific target molecule (e.g. a randomly generated molecule lacking the specifically recognized site(s)).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain (VL)” and “variable heavy chain (VH)” refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked VH-VL heterodimer which may be expressed from a nucleic acid including VH- and VL-encoding sequences either joined directly or joined by a peptide-encoding linker. Huston, et al. (1988) Proc. Nat. Acad. Sci. USA, 85: 5879-5883. While the VH and VL are connected to each as a single polypeptide chain, the VH and VL domains associate non-covalently. The scFv antibodies and a number of other structures converting the naturally aggregated, but chemically separated, F light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778).

The term “polynucleotide” refers to a deoxyribonucleotide or ribonucleotide polymer, and unless otherwise limited, includes known analogs of natural nucleotides that can function in a similar manner to naturally occurring nucleotides. The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA. The term “polynucleotide” encompasses double-stranded nucleic acid molecules, as well as single-stranded molecules. In double-stranded polynucleotides, the polynucleotide strands need not be coextensive (i.e., a double-stranded polynucleotide need not be double-stranded along the entire length of both strands).

The term “gene” refers to a polynucleotide that encodes a protein, and includes any introns, 5′ and 3′ untranslated regions, and control (e.g., promoter or enhancer) sequences.

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position. The term “substantially complementary” describes sequences that are sufficiently complementary to one another to allow for specific hybridization under stringent hybridization conditions.

The phrase “stringent hybridization conditions” generally refers to a temperature about 5° C. lower than the melting temperature (Tₘ) for a specific sequence at a defined ionic strength and pH. Exemplary stringent conditions suitable for achieving specific hybridization of most sequences are a temperature of at least about 60° C. and a salt concentration of about 0.2 molar at pH7.

“Specific hybridization” refers to the binding of a nucleic acid molecule to a target nucleotide sequence in the absence of substantial binding to other nucleotide sequences present in the hybridization mixture under defined stringency conditions. Those of skill in the art recognize that relaxing the stringency of the hybridization conditions allows sequence mismatches to be tolerated.

The phrases “an effective amount” and “an amount sufficient to” refer to amounts of a biologically active agent that produce an intended biological activity.

A “knockout mammal” is a mammal in which an endogenous gene has been disrupted by genetic engineering.

As used herein, a “wild-type mammal” refers to any mammal that expresses a natural form a gene of interest (e.g., CAP/SORBS1). A wild-type mammal can have a disruption in an irrelevant gene. Typically, a wild-type mammal expresses the protein encoded by the gene of interest at a level that is within a normal range for such mammals (e.g, of matched according to species, sex, and/or age, etc.).

The term “insulin homeostasis” refers to the state of, or tendency toward, normal (non-pathalogical) insulin levels, which vary appropriately in response to various stimuli.

The term “glucose homoestasis” refers to the state of, or tendency toward, normal (non-pathological) glucose levels, which vary appropriately in response to various stimuli.

As used herein “a disorder of insulin or glucose homeostasis” refers to any condition characterized by abnormal insulin or glucose levels and/or abnormal regulation of insulin or glucose levels. Examples of such disorder can include metabolic syndrome, pre-diabetes, diabetes, and obesity.

The term “glucose tolerance” refers to the ability of a subject to clear glucose from the blood. Glucose tolerance can be tested by administering glucose to a subject, followed by the determination of blood glucose level(s).

The term “insulin resistance” refers to the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake, whereas insulin resistance in liver reduces glucose storage, with both effects serving to elevate blood glucose. High plasma levels of insulin and glucose due to insulin resistance often leads to the metabolic syndrome and type 2 diabetes.

The term “insulin sensitivity” refers to glucose tolerance within a normal range.

A “test agent” is any agent that can be screened in the prescreening or screening assays of the invention. The test agent can be any suitable composition, including a small molecule, peptide, or polypeptide.

A test agent” “modulates” glucose tolerance or insulin resistance, when the agent increase or decreases any indicator of glucose tolerance or insulin resistance, respectively.

As used herein, the term “metabolic syndrome” refers to a combination of medical disorders that increases risk for cardiovascular disease and diabetes. Characteristics of metabolic syndrome include excess weight or obesity, high cholesterol levels, high glucose, and high blood pressure.

### III. Knockout Animals

A. In General

This invention provides animals comprising a disruption of one or both alleles of an CAP/SORBS1 gene. The CAP/SORBS1 protein modulates insulin/glucose homeostasis. The knockout animals of this invention provide good animal models for studying disorders of insulin/glucose homeostasis, such as, for example, metabolic syndrome, pre-diabetes, diabetes, and obesity. The knockout animals of this invention are further useful as systems in which to screen for various agents (e.g. drugs) that modulate insulin/glucose homeostasis and/or ameliorate on or more symptoms of a disorder of insulin/glucose homeostasis.

The invention makes use of nucleic acid sequences (transgenes) that are capable of inactivating endogenous CAP/SORBS1 genes. Such transgenes preferably contain a nucleic acid sequence (e.g., a DNA sequence) that is identical to some portion of the endogenous CAP/SORBS1 gene that is to be disrupted. Preferred transgenes of this invention also contain an insertion, deletion, or substitution of one or more nucleotides; a frameshift mutation; and/or a stop codon as compared with undisrupted alleles of the same CAP/SORBS1 gene naturally-occurring in the species.

Homologous recombination of the transgene with a CAP/SORBS1 allele disrupts the expression of that allele. Such a disruption can be by a number of mechanisms including, but not limited to, interference in initiation of transcription and/or translation, by premature termination of transcription and/or translation, and/or by production of a non-functional CAP/SORBS1 protein.

In one embodiment, such transgenes are derived by deleting nucleotides from the nucleic acid sequence encoding the functional CAP/SORBS1 gene. Although the resultant mutated nucleic acid sequence is incapable of being transcribed and/or translated into a functional CAP/SORBS1 gene product, such transgenes will have sufficient sequence homology with an endogenous CAP/SORBS1 allele of a selected non-human animal such that the transgene is capable of homologous recombination with the endogenous CAP/SORBS1 allele.

Previous studies on CAP/SORBS1 suggested the existence of various isoforms or alternatively spliced variants of this gene. At least 13 isoforms have been identified as alternative splicing products of the human SORBS1 gene. (Lin W H, et al. Cloning, mapping, and characterization of the human sorbin and SH3 domain containing 1 (SORBS1) gene: a protein associated with c-Abl during insulin signaling in the hepatoma cell line Hep3B (2001) Genomics 74:12-20; incorporated herein by reference in its entirety.) Northern blot analyses on multiple mouse tissues identified several CAP mRNA transcripts of different sizes. An anti-CAP antibody, which recognizes the C-terminal portion of CAP, was able to detect multiple proteins in Western Blot analyses, indicating multiple splicing products of the mouse gene as well. (Ribon V, Printen J A, Hoffman N G, Kay B K, Saltiel A R. A novel, multifuntional c-Cbl binding protein in insulin receptor signaling in 3T3-L1 adipocytes (1998) Mol Cell Biol. 18:872-9; incorporated herein by reference in its entirety.) Two splicing variants have been identified by a blot overlay method on subcellular fractions of rat liver with labeled 1-afadin and denoted as ponsin for interaction with 1-afadin and vinculin. (Mandai K, et al. Ponsin/SH3P12: an 1-afadin- and vinculin-binding protein localized at cell-cell and cell-matrix adherens junctions (1999) J Cell Biol. 144:1001-17; incorporated herein by reference in its entirety.) In 2003, Zhang et al. reported the cloning of 3 new splicing isoforms of CAP from mouse adipose tissue and the characterization of their protein interaction domains and subcellular localization in adipocytes. (Zhang, M., et al. Cloning and Characterization of Cbl-associated Protein Splicing Isoforms (2003) Mol. Med. 9(1-2):18-25; incorporated herein by reference in its entirety.) As those of skill in the art readily appreciate, the specific design of the transgene will determine which CAP/SORBS1 isoforms are knocked out. By knocking out individual isoforms one can examine the effect of deleting specific CAP/SORBS1 isoforms on regulation of glucose homeostasis. Furthermore, knock-in of a specific isoform in the knockout mice model provides another means of examining the specific function of the knock-in isoform.

In a preferred embodiment, transgenes are produced by ligation of an expression cassette encoding a selectable marker into the nucleic acid sequence encoding the CAP/SORBS1 gene products and/or into the nucleic acid sequence regulating transcription of the CAP/SORBS1 gene product. The cassette is preferably inserted in a location such that it replaces or disrupts regions of the encoded protein required for protein functionality. The cassette is also preferably inserted in a location such that splicing out of the cassette introduces a frameshift mutation resulting in non-functional reversions. In an exemplary embodiment, the expression cassette comprises one or more selectable marker(s), such as, e.g., β-galactosidase and/or neomycin phosphotransferase II.

Such transgenes are preferably designed for replacement of one or more exons of the endogenous CAP/SORBS1 gene. Although insertional transgenes may also be used, replacement transgenes are preferred because they significantly reduce the likelihood of secondary recombination and reversion to the wild-type CAP/SORBS1 gene.

The CAP/SORBS1 knockouts of this invention are useful themselves as models systems for a number of pathologies or can be crossed with animals exhibiting particular phenotypic traits to produce useful animal models. Such examples include, but not limited to, animals exhibiting obesity, diabetes, insulin resistance, hypertension, and high cholesterol.

B. Targeting of the Disruption: Homologous Recombination

In a particular embodiment, the present invention uses the process of homologous recombination to control the site of integration of a specific DNA sequence (transgene) into the naturally present CAP/SORBS1 sequence of an animal cell and thereby disrupt that gene and prevent normal its normal expression. Homologous recombination is described in detail by Watson (1977) In: Molecular Biology of the Gene, 3rd Ed., W.A. Benjamin, Inc., Menlo Park, Calif. In brief, homologous recombination is a natural cellular process that results in the scission of two nucleic acid molecules having identical or substantially similar (i.e., “homologous”) sequences, and the ligation of the two molecules such that one region of each initially present molecule is now ligated to a region of the other initially present molecule (Sedivy (1988) Bio-Technol., 6: 1192-1196).

Homologous recombination is exploited by a number of various methods of “gene targeting” well known to those of skill in the art (see, e.g., Mansour et al. (1988) Nature, 336: 348-352; Capecchi (1989) Trends Genet. 5: 70-76; Capecchi (1989) Science 244: 1288-1292; Capecchi et al. (1989) pages 45-52 In: Current Communications in Molecular Biology, Capecchi, M. R. (ed.), Cold Spring Harbor Press, Cold Spring Harbor, N.Y.; Frohman et al. (1989) Cell 56: 145-147). Some approaches further involve increasing the frequency of recombination between two DNA molecules by treating the introduced DNA with agents which stimulate recombination (e.g., trimethylpsoralen, UV light, etc.), however, most approaches utilize various combinations of selectable markers to facilitate isolation of the transformed cells.

One such selection method is termed positive/negative selection (PNS) (Thomas and Cappechi (1987) Cell 51: 503-512). This method involves the use of two selectable markers: one a positive selection marker such as the bacterial gene for neomycin resistance (neo); the other a negative selection marker such as the herpes virus thymidine kinase (HSV-tk) gene. Neo confers resistance to the drug G-418, while HSV-tk renders cells sensitive to the nucleoside analog gangcyclovir (GANC) or 1-(2-deoxy-2-fluoro-b-d-arabinofuranosyl)-5-iodouraci21 (FIAU). The DNA encoding the positive selection marker in the transgene (e.g., neo) is generally linked to an expression regulation sequence that allows for its independent transcription in embryonic stem (ES) cells. It is flanked by first and second sequence portions of at least a part of the CAP/SORBS1 gene.

These first and second sequence portions target the transgene to a specific allele. A second independent expression unit capable of producing the expression product for a negative selection marker, e.g., for HSV-tk is positioned adjacent to or in close proximity to the distal end of the first or second portions of the first DNA sequence. Upon transfection, some of the ES cells incorporate the transgene by random integration, others by homologous recombination between the endogenous allele and sequences in the transgene. As a result, one copy of the targeted allele is disrupted by homologous recombination with the-transgene with simultaneous loss of the sequence encoding herpes HSV-tk gene. Random integrants, which occur via the ends of the transgene, contain herpes HSV-tk and remain sensitive to GANC or FIAU. Therefore, selection, either sequentially or simultaneously with G418 and GANC enriches for transfected ES cells containing the transgene integrated into the genome by homologous recombination.

Other strategies that select for homologous recombination events but do not use PNS may also be used. For example, a promoter that is active in ES cells is operably linked to a positive selection gene such as the bacterial neo gene whose transcription unit lacks its own polyadenylation (poly-A) signal sequence. This expression unit is targeted to an exon of the endogenous CAP/SORBS1 gene. Upon homologous recombination (e.g., in the ES cell) the neo gene is transcribed independently, as above. Stable transcripts from the neo gene require the presence of a poly-A site downstream. Thus, by targeting the neo gene to an endogenous CAP/SORBS1 transcription unit, homologous recombinants are linked to the poly-A site of the targeted CAP/SORBS1 gene which permits transcription of a functional neo transcript and selection based upon resistance to G418.

It is possible that in some circumstances it will not be desirable to have an expressed antibiotic resistance gene incorporated into the knockout animal. Therefore, in certain preferred embodiments, one or more genetic elements are included in the knockout construct that permit the antibiotic resistance gene to be excised once the construct has undergone homologous recombination with the CAP/SORBS1 gene.

The FLP/FRT recombinase system from yeast represents one such set of genetic elements (O'Gorman et al. (1991) Science 251, 1351-1355). FLP recombinase is a protein of approximately 45 kD molecular weight. It is encoded by the FLP gene of the 2 micron plasmid of the yeast Saccharomyces cerevisiae. The protein acts by binding to the FLP Recombinase target site, or FRT; the core region of the FRT is a DNA sequence of approximately 34 bp. FLP can mediate several kinds of recombination reactions including excision, insertion, and inversion, depending on the relative orientations of flanking FRT sites. If a region of DNA is flanked by direct repeats of the FRT, FLP will act to excise the intervening DNA, leaving only a single FRT. FLP has been shown to function in a wide range of systems, including in the cultured mammalian cell lines CV-1 and F9, (O'Gorman et al. supra; and in mouse ES cells, Jung et al. (1993) Science 259: 984).

The methods described herein are capable of mutating both alleles of the cell's CAP/SORBS1 gene; however, since the frequency of such dual mutational events is the square of the frequency of a single mutational event, cells having mutations in both of their CAP/SORBS1 alleles will be only a very small proportion of the total population of mutated cells. It is possible to readily identify (for example through the use of Southern hybridization or other methods) whether the mutational events are single-allele or dual-allele events. Animals having a mutational event in a single allele may be cross-bred to produce homozygous animals (having the disruption in both alleles) if the disruption becomes incorporated in the germ line.

In a preferred embodiment, the nucleic acid molecule(s) that are to be introduced into the recipient cell contain a region of homology with a region of the CAP/SORBS1 gene. In a preferred embodiment, the nucleic acid molecule will contain two regions having homology with the cell's CAP/SORBS1 gene. These “regions of homology” will preferably flank the precise sequence whose incorporation into the CAP/SORBS1 gene is desired.

The nucleic acid molecule(s) may be single stranded, but are preferably double stranded. The molecule(s) may be introduced to the cell as DNA molecules, as one or more RNA molecules which may be converted to DNA by reverse transcriptase or by other means.

C. Transformation of Cells

To produce the knockout animal, cells are transformed with the construct (e.g., transgene) described above. As used herein, the term “transformed” is defined as introduction of exogenous DNA into the target cell by any means known to the skilled artisan. These methods of introduction can include, without limitation, transfection, microinjection, infection (with, for example, retroviral-based vectors), electroporation and microballistics. The term “transformed,” unless otherwise indicated, is not intended herein to indicate alterations in cell behavior and growth patterns accompanying immortalization, density-independent growth, malignant transformation or similar acquired states in culture.

To create animals having a particular gene inactivated in all cells, it is preferable to introduce a knockout construct into the germ cells (sperm or eggs, i.e., the “germ line”) of the desired species. Genes or other DNA sequences can be introduced into the pronuclei of fertilized eggs by microinjection or other methods as described below. Following pronuclear fusion, the developing embryo may carry the introduced gene in all its somatic and germ cells since the zygote is the mitotic progenitor of all cells in the embryo. Since targeted insertion of a knockout construct is a relatively rare event, it is desirable to generate and screen a large number of animals when employing such an approach. Because of this, it can be advantageous to work with the large cell populations and selection criteria that are characteristic of cultured cell systems. However, for production of knockout animals from an initial population of cultured cells, it is preferred that a cultured cell containing the desired knockout construct be capable of generating a whole animal. This is generally accomplished by placing the cell into a developing embryo environment of some sort.

Cells capable of giving rise to at least several differentiated cell types are hereinafter termed “pluripotent” cells. Pluripotent cells capable of giving rise to all cell types of an embryo, including germ cells, are hereinafter termed “totipotent” cells. Totipotent murine cell lines (embryonic stem, or “ES” cells) have been isolated by culture of cells derived from very young embryos (blastocysts). Such cells are capable, upon incorporation into an embryo, of differentiating into all cell types, including germ cells, and can be employed to generate animals lacking a functional CAP/SORBS1 gene. That is, cultured ES cells can be transformed with a knockout construct, as described herein, and cells selected in which the CAP/SORBS1 gene is inactivated through insertion of the construct within the CAP/SORBS1 gene.

1. Microinjection Methods

The transgenic non-human animals of the invention are produced by introducing transgenes into the germline of the non-human animal. Embryonic target cells at various developmental stages can be used to introduce transgenes. Different methods are used depending on the stage of development of the embryonic target cell.

Microinjection is one preferred method for transformation of a zygote. In the mouse, the male pronucleus reaches the size of approximately 20 micrometers in diameter which allows reproducible injection of 1-2 μl of DNA solution. The use of zygotes as a target for gene transfer has a major advantage in that in most cases the injected DNA will be incorporated into the host gene before the first cleavage (Brinster et al. (1985) Proc. Natl. Acad. Sci. USA 82, 4438-4442). As a consequence, all cells of the transgenic non-human animal will carry the incorporated transgene. This will, in general, also be reflected in the efficient transmission of the transgene to offspring of the founder since 50% of the germ cells will harbor the transgene.

The gene sequence being introduced need not be incorporated into any kind of self-replicating plasmid or virus (Jaenisch, (1988) Science, 240: 1468-1474). Indeed, the presence of vector DNA has been found, in many cases, to be undesirable (Hammer et al. (1987) Science 235: 53; Chada et al. (1986) Nature 319: 685; Kollias et al., (1986) Cell 46: 89; Shani, (1986) Molec, Cell, Biol. 6: 2624 (1986); Chada, et al. (1985) Nature, 314: 377; Townes et al. (1985) EMBO J. 4: 1715).

Once the DNA molecule has been injected into the fertilized egg cell, the cell is implanted into the uterus of a recipient female, and allowed to develop into an animal. Since all of the animal's cells are derived from the implanted fertilized egg, all of the cells of the resulting animal (including the germ line cells) contain the introduced gene sequence. If, as occurs in about 30% of events, the first cellular division occurs before the introduced gene sequence has integrated into the cell's genome, the resulting animal will be a chimeric animal.

By breeding and inbreeding such animals, it is possible to routinely produce heterozygous and homozygous transgenic animals. Despite any unpredictability in the formation of such transgenic animals, the animals have generally been found to be stable, and to be capable of producing offspring that retain and express the introduced gene sequence.

The success rate for producing transgenic animals is greatest in mice. Approximately 25% of fertilized mouse eggs into which DNA has been injected, and which have been implanted in a female, will become transgenic mice. A number of other transgenic animals have also been produced. These include rabbits, sheep, cattle, and pigs (Jaenisch (1988) Science 240: 1468-1474; Hammer et al., (1986) J. Animal. Sci, 63: 269 Hammer et al. (1985) Nature 315: 680; Wagner et al., (1984) Theriogenology 21: 29).

2. Retroviral Methods

Retroviral infection can also be used to introduce a transgene into a non-human animal. The developing non-human embryo can be cultured in vitro to the blastocyst stage. During this time, the blastomeres can be targets for retroviral infection (Jaenich (1976) Proc. Natl. Acad. Sci. USA 73: 1260-1264). Efficient infection of the blastomeres is obtained by enzymatic treatment to remove the zona pellucida (Hogan, et al. (1986) In Manipulating the Mouse Embryo, Cold Spring Harbor Laboratory Press, Cold Spring Harbor, N.Y.). The viral vector system used to introduce the transgene is typically a replication-defective retrovirus carrying the transgene (Jahner, et al. (1985) Proc. Natl. Acad. Sci. USA 82, 6927-6931; Van der Putten, et al. (1985) Proc. Natl. Acad. Sci., USA, 82, 6148-6152). Transfection is easily and efficiently obtained by culturing the blastomeres on a monolayer of virus-producing cells (Van der Putten, supra; Stewart et al. (1987) EMBO J., 6: 383-388). Alternatively, infection can be performed at a later stage. Virus or virus-producing cells can be injected into the blastocoele (Jahner et al. (1982) Nature, 298: 623-628). Most of the founders will be mosaic for the transgene since incorporation occurs only in a subset of the cells, which formed the transgenic non-human animal. Further, the founder may contain various retroviral insertions of the transgene at different positions in the genome which generally will segregate in the offspring. In addition, it is also possible to introduce transgenes into the germ line, albeit with low efficiency, by intrauterine retroviral infection of the midgestation embryo (Jahner et al. (1982) supra).

3. ES Cell Implantation

A third and preferred target cell for transgene introduction is the embryonic stem cell (ES). ES cells are obtained from pre-implantation embryos cultured in vitro and fused with embryos (Evans, et al. (1981) Nature, 292: 154-156; Bradley, et al. (1984) Nature, 309: 255-258; Gossler, et al. (1986) Proc. Natl. Acad. Sci., USA, 83: 9065-9069; and Robertson, et al. (1986) Nature, 322: 445-448). Transgenes can be efficiently introduced into the ES cells a number of means well known to those of skill in the art. Such transformed ES cells can thereafter be combined with blastocysts from a non-human animal. The ES cells thereafter colonize the embryo and contribute to the germ line of the resulting chimeric animal (for a review see Jaenisch (1988) Science, 240: 1468-1474).

The DNA molecule containing the desired gene sequence may be introduced into the pluripotent cell by any method which will permit the introduced molecule to undergo recombination at its regions of homology. Transgenes can be efficiently introduced into the ES cells by DNA transfection or by retrovirus-mediated transduction.

In a particular embodiment, the DNA is introduced by electroporation (Toneguzzo et al., (1988) Nucleic Acids Res., 16: 5515-5532; Quillet et al. (1988) J. Immunol., 141: 17-20; Machy et al. (1988) Proc. Natl. Acad. Sci., USA, 85: 8027-8031). After permitting the introduction of the DNA molecule(s), the cells are cultured under conventional conditions, as are known in the art.

In order to facilitate the recovery of those cells that have received the DNA molecule containing the desired gene sequence, it is preferable to introduce the DNA containing the desired gene sequence in combination with a second gene sequence that would contain a detectable marker gene sequence. Where it is only desired to introduce a disruption into a gene, the DNA sequence containing the detectable marker sequence may itself comprise the disruption. For the purposes of the present invention, any gene sequence whose presence in a cell permits one to recognize and clonally isolate the cell may be employed as a detectable (selectable) marker gene sequence.

In one embodiment, the presence of the detectable (selectable) marker sequence in a recipient cell is recognized by hybridization, by detection of radiolabelled nucleotides, or by other assays of detection which do not require the expression of the detectable marker sequence. In one embodiment, such sequences are detected using polymerase chain reaction (PCR) or other DNA amplification techniques to specifically amplify the DNA marker sequence (Mullis et al., (1986) Cold Spring Harbor Symp. Quant. Biol. 51: 263-273; Erlich et al. EP 50,424; EP 84,796, EP 258,017 and EP 237,362; Mullis EP 201,184; Mullis et al., U.S. Pat. No. 4,683,202; Erlich U.S. Pat. No. 4,582,788; and Saiki et al. U.S. Pat. No. 4,683,194).

Most preferably, however, the detectable marker gene sequence will be expressed in the recipient cell and will result in a selectable phenotype. Selectable markers are well known to those of skill in the art. Some examples include the hprt gene (Littlefield Science 145:709-710), the thymidine kinase gene of herpes simplex virus (Giphart-Gassler et al. (1989) Mutat, Res., 214: 223-232), the nDtII gene (Thomas et al. (1987) Cell, 51: 503-512; Mansour et al. (1988) Nature 336: 348-352), or other genes which confer resistance to amino acid or nucleoside analogues, or antibiotics, etc.

Thus, for example, cells that express an active HPRT enzyme are unable to grow in the presence of certain nucleoside analogues (such as 6-thioguanine, 8-azapurine, etc.), but are able to grow in media supplemented with HAT (hypoxanthine, aminopterin, and thymidine). Conversely, cells which fail to express an active HPRT enzyme are unable to grow in media containing HATG, but are resistant to analogues such as 6-thioguanine, etc. (Littlefield (1964) Science, 145: 709-710). Cells expressing active thymidine kinase are able to grow in media containing HAT, but are unable to grow in media containing nucleoside analogues such as bromo-deoxyuridine (Giphart-Gassler et al. (1989) Mutat. Res. 214: 223-232). Cells containing an active HSV-tk gene are incapable of growing in the presence of gangcylovir or similar agents.

The detectable marker gene may also be any gene that can compensate for a recognizable cellular deficiency. Thus, for example, the gene for HPRT could be used as the detectable marker gene sequence when employing cells lacking HPRT activity. This agent is an example of agents may be used to select mutant cells, or to “negatively select” for cells which have regained normal function.

In preferred embodiments, the chimeric or transgenic animal cells of the present invention are prepared by introducing one or more DNA molecules into a precursor pluripotent cell, most preferably an ES cell, or equivalent (Robertson (1989) pages 39-44 In: Current communications in Molecular Biology, Capecchi, M. R. (ed.), Cold Spring Harbor Press, Cold Spring Harbor, N.Y.—The term “precursor” is intended to denote only that the pluripotent cell is a precursor to the desired (“transfected”) pluripotent cell which is prepared in accordance with the teachings of the present invention. The pluripotent (precursor or transfected) cell may be cultured in vivo, in a manner known in the art (Evans et al., (1981) Nature 292: 154-156) to form a chimeric or transgenic animal. The transfected cell, and the cells of the embryo that it forms upon introduction into the uterus of a female are herein referred to respectively, as “embryonic stage” ancestors of the cells and animals of the present invention.

Any ES cell may be used in accordance with the present invention. It is, however, preferred to use primary isolates of ES cells. Such isolates may be obtained directly from embryos such as the CCE cell line disclosed by Robertson, E. J., In: Current Communications in Molecular Biology, Capecchi, M. R. (ed.), Cold Spring Harbor Press, Cold Spring Harbor, N.Y. (1989), pp. 39-44), or from the clonal isolation of ES cells from the CCE cell line (Schwartzberg et al. (1989) Science 212: 799-803). Such clonal isolation may be accomplished according to the method of Robertson (1987) In: Teratocarcinomas and Embryonic Stem Cells: A Practical Approach, E. J. Robertson, Ed., IRL Press, Oxford. The purpose of such clonal propagation is to obtain ES cells that have a greater efficiency for differentiating into an animal. Clonally selected ES cells are approximately 10-fold more effective in producing transgenic animals than the progenitor cell line CCE. An example of ES cell lines which have been clonally derived from embryos are the ES cell lines, AB1 (hprt+) or AB2.1 (hprt−).

The ES cells are preferably cultured on stromal cells (such as STO cells (especially SNL76/7 STO cells) and/or primary embryonic G418 R fibroblast cells) as described by Robertson, supra. Methods for the production and analysis of chimeric mice are well known to those of skill in the art (see, e.g., Bradley (1987) pages 113-151 In: Teratocarcinomas and Embryonic Stem Cells; A Practical Approach, E. J. Robertson, ed., IRL Press, Oxford). The stromal (and/or fibroblast) cells serve to eliminate the clonal overgrowth of abnormal ES cells. Most preferably, the cells are cultured in the presence of leukocyte inhibitory factor (“lif”) (Gough et al. (1989) Reprod. Fertil., 1: 281-288; Yamamori et al. (1989) Science, 246: 1412-1416). Since the gene encoding lif has been cloned (Gough, et al. supra.), it is especially preferred to transform stromal cells with this gene, by means known in the art, and to then culture the ES cells on transformed stromal cells that secrete lif into the culture medium.

ES cell lines may be derived or isolated from any species (for example, chicken, fish, etc.), although cells derived or isolated from mammals such as rodents, rabbits, sheep, goats, pigs, cattle, primates and humans are preferred. Cells derived from rodents (i.e., mouse, rat, hamster, etc.) are particularly preferred.

In fact, ES cell lines have been derived for mice and pigs as well as other animals (see, e.g., Robertson, Embryo-Derived Stem Cell Lines. In: Teratocarcinomas and Embryonic Stem Cells: A Practical Approach (E. J. Robertson, ed.), IRL Press, Oxford (1987); PCT Publication No. WO/90/03432; PCT Publication No. 94/26884. Generally these cells lines must be propagated in a medium containing a differentiation-inhibiting factor (DIF) to prevent spontaneous differentiation and loss of mitotic capability. Leukemia Inhibitory Factor (LIF) is particularly useful as a DIF. Other DIFs useful for prevention of ES cell differentiation include, without limitation, Oncostatin M (Gearing and Bruce (1992) The New Biologist 4: 61-65), interleukin 6 (IL-6) with soluble IL-6 receptor (sIL-6R) (Taga et al. (1989) Cell 58: 573-581), and ciliary neurotropic factor (CNTF) (Conover et al. (1993) Development 19: 559-565). Other known cytokines may also function as appropriate DIFs, alone or in combination with other DIFs.

As a useful advance in maintenance of ES cells in an undifferentiated state, a novel variant of LIF (T-LIF) has been identified (see U.S. Pat. No. 5,849,991). In contrast to the previously identified forms of LIF which are extracellular, T-LIF is intracellularly localized. The transcript was cloned from murine ES cells using the RACE technique (Frohman et al. (1988) Proc. Natl. Acad. Sci., USA, 85: 8998-9002) and subjected to sequence analysis. Analysis of the obtained nucleic acid sequence and deduced amino acid sequence indicates that T-LIF is a truncated form of the LIF sequence previously reported in the literature. Expression of the T-LIF nucleic acid in an appropriate host cell yields a 17 kD protein that is unglycosylated. This protein is useful for inhibiting differentiation of murine ES cells in culture.

D. Production of Transgenic Animals via Somatic Cell Nuclear Transfer

Production of the knockout animals of this invention is not dependent on the availability of ES cells. In various embodiments, knockout animals of this invention can be produced using methods of somatic cell nuclear transfer. In preferred embodiments using such an approach, a somatic cell is obtained from the species in which the CAP/SORBS1gene is to be knocked out. The cell is transfected with a construct that introduces a disruption in the CAP/SORBS1 gene (e.g., via heterologous recombination) as described herein. Cells harboring a knocked-out CAP/SORBS1 are selected as described herein. The nucleus of such cells harboring the knockout is then placed in an unfertilized enucleated egg (e.g., eggs from which the natural nuclei have been removed by microsurgery). Once the transfer is complete, the recipient eggs contain a complete set of genes, just as they would if they had been fertilized by sperm. The eggs are then cultured for a period before being implanted into a host mammal (of the same species that provided the egg) where they are carried to term, culminating in the birth of a transgenic animal comprising a nucleic acid construct containing one or more disrupted CAP/SORBS1 genes

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Campbell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrombin III gene by somatic cell nuclear transfer has been reported (Baguisi et al. (1999) Nature Biotechnology 17: 456-461).

Using methods of nuclear transfer as describe in these and other references, cell nuclei derived from differentiated fetal or adult, mammalian cells are transplanted into enucleated mammalian oocytes of the same species as the donor nuclei. The nuclei are reprogrammed to direct the development of cloned embryos, which can then be transferred into recipient females to produce fetuses and offspring, or used to produce cultured inner cell mass (CICM) cells. The cloned embryos can also be combined with fertilized embryos to produce chimeric embryos, fetuses and/or offspring.

Somatic cell nuclear transfer also allows simplification of transgenic procedures by working with a differentiated cell source that can be clonally propagated. This eliminates the need to maintain the cells in an undifferentiated state, thus, genetic modifications, both random integration and gene targeting, are more easily accomplished. Also by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report. Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

Differentiated mammalian cells are those cells that are past the early embryonic stage. More particularly, the differentiated cells are those from at least past the embryonic disc stage (e.g., day 10 of bovine embryogenesis). The differentiated cells may be derived from ectoderm, mesoderm or endoderm.

Mammalian cells, including human cells, may be obtained by well known methods. Mammalian cells useful in the present invention include, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells, etc. Moreover, the mammalian cells used for nuclear transfer may be obtained from different organs, e.g., skin, lung, pancreas, liver, stomach, intestine, heart, reproductive organs, bladder, kidney, urethra and other urinary organs, etc. These are just examples of suitable donor cells. Suitable donor cells, i.e., cells useful in the subject invention, may be obtained from any cell or organ of the body. This includes all somatic or germ cells.

Fibroblast cells are an ideal cell type because they can be obtained from developing fetuses and adult animals in large quantities. Fibroblast cells are differentiated somewhat and, thus, were previously considered a poor cell type to use in cloning procedures. Importantly, these cells can be easily propagated in vitro with a rapid doubling time and can be clonally propagated for use in gene targeting procedures.

As indicated above, once the CAP/SORBS1 gene has been knocked out in a somatic cell, the nucleus is transferred to an oocyte, preferably to a mammalian oocyte. Suitable mammalian sources for oocytes include, but are not limited to, rodent, equine, bovine, porcine, lagomorph, feline, canine, murine, caprine, ovine, and non-human primate cells. Methods for isolation of oocytes are well known in the art.

The oocytes are generally matured in vitro before they are used as recipient cells for nuclear transfer. In preferred embodiments, this process generally involves collecting immature (prophase I) oocytes from mammalian ovaries, e.g., bovine ovaries obtained at a slaughterhouse, and maturing the oocytes in a maturation medium prior to until the oocyte attains the metaphase II stage, which in the case of bovine oocytes generally occurs about 18-24 hours post-aspiration. This period of time is known as the “maturation period.”

Metaphase II stage oocytes, which have been matured in vivo have also been successfully used in nuclear transfer techniques. Essentially, mature metaphase II oocytes are collected surgically from either non-superovulated or superovulated mammals (e.g., cows or heifers 35 to 48 hours) past the onset of estrus or past the injection of human chorionic gonadotropin (hCG) or similar hormone.

In general, successful mammalian embryo cloning practices use the metaphase II stage oocyte as the recipient oocyte because at this stage it is believed that the oocyte can be, or is, sufficiently “activated” to treat the introduced nucleus as it does a fertilizing sperm. In domestic animals, and especially cattle, the oocyte activation period generally ranges from about 16-52 hours, preferably about 28-42 hours post-aspiration.

For example, immature oocytes may be washed in HEPES buffered hamster embryo culture medium (HECM), as described in Seshagine et al. (1989) Biol. Reprod., 40, 544-606, and then placed into drops of maturation medium consisting of 50 microliters of tissue culture medium (TCM) 199 containing 10% fetal calf serum, which contains appropriate gonadotropins such as luteinizing hormone (LH) and follicle stimulating hormone (FSH), and estradiol under a layer of lightweight paraffin or silicon at 39° C.

After a fixed time maturation period, which ranges from about 10 to 40 hours, and preferably about 16-18 hours, the oocytes will be enucleated. Prior to enucleation the oocytes are preferably be removed and placed in HECM containing 1 milligram per milliliter of hyaluronidase prior to removal of cumulus cells. This may be effected by repeated pipetting through very fine bore pipettes or by vortexing briefly. The stripped oocytes are then screened for polar bodies, and the selected metaphase II oocytes, as determined by the presence of polar bodies, are then used for nuclear transfer. Enucleation follows.

Enucleation may be effected by known methods, such as described in U.S. Pat. No. 4,994,384. For example, metaphase II oocytes are either placed in HECM, optionally containing 7.5 μg/ml cytochalasin B, for immediate enucleation, or may be placed in a suitable medium, for example an embryo culture medium such as CR1aa, plus 10% estrus cow serum, and then enucleated later, preferably not more than 24 hours later, and more preferably 16-18 hours later.

Enucleation can also be accomplished microsurgically, e.g., using a micropipette to remove the polar body and the adjacent cytoplasm. The oocytes can then be screened to identify those of which have been successfully enucleated. This screening can be effected by staining the oocytes with 1 μg/ml 33342 Hoechst dye in HECM and then viewing the oocytes under ultraviolet irradiation for less than 10 seconds. The oocytes that have been successfully enucleated can then be placed in a suitable culture medium, e.g., CR1aa plus 10% serum.

In somatic cell nuclear transfer, the recipient oocytes are preferably enucleated at a time ranging from about 10 hours to about 40 hours after the initiation of in vitro maturation, more preferably from about 16 hours to about 24 hours after initiation of in vitro maturation, and most preferably about 16-18 hours after initiation of in vitro maturation.

A single mammalian cell of the same species as the enucleated oocyte is then transferred into the perivitelline space of the enucleated oocyte used to produce the nuclear transfer unit (NT unit). The mammalian cell and the enucleated oocyte is used to produce NT units according to methods known in the art. For example, the cells can be fused by electrofusion.

Electrofusion is accomplished by providing a pulse of electricity that is sufficient to cause a transient and brief breakdown of the plasma membrane. If two adjacent membranes are induced to breakdown and upon reformation the lipid bilayers intermingle, small channels open between the two cells. Due to the thermodynamic instability of such a small opening, it enlarges until the two cells become one. Reference is made to U.S. Pat. No. 4,997,384 by Prather et al., for a further discussion of this process. A variety of electrofusion media can be used including, e.g., sucrose, mannitol, sorbitol and phosphate buffered solution. Fusion can also be accomplished using Sendai virus as a fusogenic agent (Graham (1969) Inot. Symp. Monogr., 9:19).

Also, in some cases (e.g., with small donor nuclei) it may be preferable to inject the nucleus directly into the oocyte rather than using electroporation fusion. Such techniques are disclosed, for example in Collas and Barnes (1994) Mol. Reprod. Dev., 38:264-267.

After fusion, the resultant fused NT units are then placed in a suitable medium until activation, e.g., CR1aa medium. Typically activation will be effected shortly thereafter, typically less than 24 hours later, and preferably about 4-9 hours later.

The NT unit may be activated by known methods. Such methods include, e.g., culturing the NT unit at sub-physiological temperature, in essence by applying a cold, or actually cool temperature shock to the NT unit. This may be most conveniently done by culturing the NT unit at room temperature, which is cold relative to the physiological temperature conditions to which embryos are normally exposed.

Alternatively, activation may be achieved by application of known activation agents. For example, penetration of oocytes by sperm during fertilization has been shown to activate perfusion oocytes to yield greater numbers of viable pregnancies and multiple genetically identical calves after nuclear transfer. Also, treatments such as electrical and chemical shock may be used to activate NT embryos after fusion. Suitable oocyte activation methods are the subject of U.S. Pat. No. 5,496,720.

Additionally, activation can be effected by simultaneously or sequentially increasing levels of divalent cations in the oocyte, and/or reducing phosphorylation of cellular proteins in the oocyte. This is generally effected by introducing divalent cations (e.g., magnesium, strontium, barium or calcium, preferably in the form of an ionophore) into the oocyte cytoplasm. Other methods of increasing divalent cation levels include the use of electric shock, treatment with ethanol and treatment with caged chelators.

Phosphorylation can be reduced by known methods, e.g., by the addition of kinase inhibitors, e.g., serine-threonine kinase inhibitors, such as 6-dimethyl-aminopurine, staurosporine, 2-aminopurine, and sphingosine. Alternatively, phosphorylation of cellular proteins can be inhibited by introduction of a phosphatase into the oocyte, e.g., phosphatase 2A and phosphatase 2B.

In one embodiment, NT activation is effected by briefly exposing the fused NT unit to a TL-HEPES medium containing 5 μM ionomycin and 1 mg/ml BSA, followed by washing in TL-HEPES containing 30 mg/ml BSA within about 24 hours after fusion, and preferably about 4 to 9 hours after fusion.

The activated NT units can then be cultured in a suitable in vitro culture medium until the generation of CICM cells and cell colonies. Culture media suitable for culturing and maturation of embryos are well known in the art. Examples of known media, which may be used for bovine embryo culture and maintenance, include, but are not limited to, Ham's F-10+10% fetal calf serum (FCS), Tissue Culture Medium-199 (TCM-199)+10% fetal calf serum, Tyrodes-Albumin-Lactate-Pyruvate (TALP), Dulbecco's Phosphate Buffered Saline (PBS), Eagle's and Whitten's media. One of the most common media used for the collection and maturation of oocytes is TCM-199, and a 1 to 20% serum supplement, including fetal calf serum, newborn serum, estrual cow serum, lamb serum or steer serum. A preferred maintenance medium includes TCM-199 with Earl salts, 10% fetal calf serum, 0.2 mM Na pyruvate and 50 μg/ml gentamicin sulphate. Any of the above may also involve co-culture with a variety of cell types such as granulosa cells, oviduct cells, BRL cells and uterine cells and STO cells.

Another maintenance medium is described in U.S. Pat. No. 5,096,822. This embryo medium, named CR1, contains the nutritional substances necessary to support an embryo. CR1 contains hemicalcium L-lactate in amounts ranging from 1.0 mM to 10 mM, preferably 1.0 mM to 5.0 mM. Hemicalcium L-lactate is L-lactate with a hemicalcium salt incorporated thereon. Hemicalcium L-lactate is significant in that a single component satisfies two major requirements in the culture medium: (i) the calcium requirement necessary for compaction and cytoskeleton arrangement; and (ii) the lactate requirement necessary for metabolism and electron transport. Hemicalcium L-lactate also serves as valuable mineral and energy source for the medium necessary for viability of the embryos.

Advantageously, CR1 medium does not contain serum, such as fetal calf serum, and does not require the use of a co-culture of animal cells or other biological media, i.e., media comprising animal cells such as oviductal cells. Biological media can sometimes be disadvantageous in that they may contain microorganisms or trace factors which may be harmful to the embryos and which are difficult to detect, characterize and eliminate.

Examples of the main components in CR1 medium include hemicalcium L-lactate, sodium chloride, potassium chloride, sodium bicarbonate and a minor amount of fatty-acid free bovine serum albumin (Sigma A-6003). Additionally, a defined quantity of essential and non-essential amino acids may be added to the medium. CR1 with amino acids is known by the abbreviation “CR1aa.”

In one embodiment, the activated NT embryos unit are placed in CR1aa medium containing 1.9 mM DMAP for about 4 hours followed by a wash in HECM and then cultured in CR1aa containing BSA.

For example, the activated NT units may be transferred to CR1aa culture medium containing 2.0 mM DMAP (Sigma) and cultured under ambient conditions, e.g., about 38.5° C., 5% CO₂ for a suitable time, e.g., about 4 to 5 hours.

Afterward, the cultured NT unit or units are preferably washed and then placed in a suitable media, e.g., CR1aa medium containing 10% FCS contained in well plates which preferably contain a suitable confluent feeder layer. Suitable feeder layers include, by way of example, fibroblasts and epithelial cells, e.g., fibroblasts and uterine epithelial cells derived from ungulates, chicken fibroblasts, murine (e.g., mouse or rat) fibroblasts, STO and SI-m220 feeder cell lines, and BRL cells. In one embodiment, the feeder cells comprise mouse embryonic fibroblasts.

The NT units are cultured on the feeder layer until the NT units reach a size suitable for transferring to a recipient female, or for obtaining cells which may be used to produce CICM cells or cell colonies. Preferably, these NT units will be cultured until at least about 2 to 400 cells, more preferably about 4 to 128 cells, and most preferably at least about 50 cells. The culturing will be effected under suitable conditions, i.e., about 38.5° C. and 5% CO₂, with the culture medium changed in order to optimize growth typically about every 2-5 days, preferably about every 3 days.

The methods for embryo transfer and recipient animal management for somatic cell nuclear transfer are standard procedures used in the embryo transfer industry. Synchronous transfers are important for success of the somatic cell nuclear transfer, i.e., the stage of the NT embryo is in synchrony with the estrus cycle of the recipient female. This advantage and how to maintain recipients are reviewed in Siedel, G. E., Jr. (“Critical review of embryo transfer procedures with cattle” in Fertilization and Embryonic Development in Vitro (1981) L. Mastroianni, Jr. and J. D. Biggers, ed., Plenum Press, New York, N.Y., page 323).

For production of CICM cells and cell lines, after NT units of the desired size are obtained, the cells are mechanically removed from the zone and are then used. This is preferably effected by taking the clump of cells which comprise the NT unit, which typically will contain at least about 50 cells, washing such cells, and plating the cells onto a feeder layer, e.g., irradiated fibroblast cells. Typically, the cells used to obtain the stem cells or cell colonies will be obtained from the innermost portion of the cultured NT unit which is preferably at least 50 cells in size. However, NT units of smaller or greater cell numbers as well as cells from other portions of the NT unit may also be used to obtain ES cells and cell colonies. The cells are maintained in the feeder layer in a suitable growth medium, e.g., alpha MEM supplemented with 10% FCS and 0.1 mM β-mercaptoethanol (Sigma) and L-glutamine. The growth medium is changed as often as necessary to optimize growth, e.g., about every 2-3 days.

E. Other Non-Human Animals

Having shown that disruption of the CAP/SORBS1 gene can modulate insulin/glucose homestasis and that CAP/SORBS1-deficient animals are viable, one of skill will recognize that there are a wide number of animals including natural and transgenic animals that have other desirable phenotypes and that can be used to practice the invention. Preferred animals are mammals including, but not limited to, rodents (e.g, murines), equines, bovines, porcines, lagomorphs, felines, canines, caprines, ovines, non-human primates, and the like.

Zygotes or ES cells from the CAP/SORBS1 knockouts of this invention can be used as embryonic target cells for introduction of other heterologous genes or knockout constructs. Alternatively somatic cells can be used as targets for the introduction of various heterologous expression cassettes or knockout constructs.

In other embodiments, the knockout animals of this invention can be can be cross-bred with other animals exhibiting various natural or induced pathologies. In various embodiments, the knockout animals of this invention are crossed with animals having one or more knockouts other than the CAP/SORBS1 knockout.

In certain preferred embodiments, a transgenic non-human animal is bred that that includes a deficiency in CAP/SORBS1 expression (e.g., a heterozygous or homozygous CAP/SORBS1 knockout) and a deficiency in a second recombinantly disrupted gene.

Preferred variants include, but are not limited to animals produced by crossing an animal with a disruption in the CAP/SORBS1 gene to an animal that has a natural, or bred, or recombinantly introduced, predisposition to a disorder of insulin/glucose homeostasis.

One of skill will recognize that targeting of a transgene to a CAP/SORBS1 allele in another species is facilitated by knowledge of the sequence of CAP/SORBS1 gene in the subject species in order to incorporate the appropriate targeting sequences. The structure and function of the CAP/SORBS1 genes other species are well known or can easily be ascertained using well known techniques by those of skill in the art.

For example, sequences from the mouse, hamster or human may be used as probes to identify the corresponding gene in other species using techniques well known to those of skill in the art. Thus, for example, a genomic or cDNA library for the subject species may be produced following published procedures (see, for example, Young et al. (1983) Proc. Natl. Acad. Sci., USA, 170: 827-842; Frischauf et al. (1983) J. Mol. Biol. 170: 827-842; Sambrook et al. (1989) Molecular Cloning: A Laboratory Manual (2nd Ed., Vols. 1-3, Cold Spring Harbor Laboratory). The library may screened (i.e., in a Southern Blot) under conditions of reduced stringency with appropriate probes to segments of the CAP/SORBS1 gene. Once segments of the CAP/SORBS1 gene in the subject species are identified, sequencing of the entire gene may be accomplished using routine methods well known to those of skill in the art (see, for example, Sambrook supra).

Once the target sequences in the CAP/SORBS1 gene of the other species are identified, creation of the disrupting transgene is routine to one of skill as described above. One may simply insert a disrupting marker, or alternatively one may introduce various insertions, deletions, or mutations as described above in section. Transformation of the subject organism may be accomplished using one of the methods described above.

### IV. Methods of Screening for Agents that Modulate Insulin/Glucose Homeostasis

The role of CAP/SORBS1 in insulin/glucose homeostasis makes this molecule an attractive target for agents useful in the prophylaxis or treatment of a disorder of insulin/glucose homeostasis. Accordingly, the invention provides prescreening and screening methods aimed at identifying such agents. Test agents can be prescreened, for example, based on binding to a CAP/SORBS1 polypeptide or on binding to a CAP/SORBS1 polynucleotide. In addition, the invention provides screening methods based on screening for effects on the levels of CAP/SORBS1 or a polynucleotide encoding CAP/SORBS1 (e.g., CAP/SORBS1 mRNA) or for effects on CAP/SORBS1 activity. Agents that increase levels and/or activity of CAP/SORBS1 polypeptides or polynucleotides corresponding to the sequences affected in the knockout mice exemplified herein are useful, in particular, in the prophylaxis or treatment of diabetes.

The prescreening/screening methods of the invention are generally, although not necessarily, carried out in vitro. Accordingly, screening assays are generally carried out, for example, using purified or partially purified components in cell lysates or fractions thereof, in cultured cells, or in a biological sample, such as a tissue or a fraction thereof.

A. Prescreening Based on Binding to CAP/SORBS1

The invention provides a prescreening method based on assaying test agents for specific binding to a CAP/SORBS1 polypeptide. Agents that specifically bind to CAP/SORBS1 have the potential to modulate function and thereby modulate insulin/glucose homeostasis.

In one embodiment, therefore, a prescreening method of the invention entails contacting a test agent with a CAP/SORBS1 polypeptide. The CAP/SORBS1 polypeptide can be a full-length polypeptide or a fragment thereof. The CAP/SORBS1 polypeptide can also be a fusion polypeptide, including the full CAP/SORBS1 amino acid sequence, or a portion thereof, linked to one or more other amino acid sequences. CAP/SORBS1 polypeptides useful in the method typically have a wild-type amino acid sequence; however, the method can be carried out using amino acid variants and/or polypeptides that are otherwise modified. Any such amino acid sequence variations or modifications typically leave at least a segment of CAP/SORBS1 amino acid sequence free to interact with the test agent.

Specific binding of the test agent to the CAP/SORBS1 polypeptide is then determined. If specific binding is detected, the test agent is selected as a potentially useful in the prophylaxis or treatment of a disorder of insulin/glucose homeostasis.

Such prescreening is generally most conveniently accomplished with a simple in vitro binding assay. Means of assaying for specific binding of a test agent to a polypeptide are well known to those of skill in the art. In preferred binding assays, the polypeptide is immobilized and exposed to a test agent (which can be labeled), or alternatively, the test agent(s) are immobilized and exposed to the polypeptide (which can be labeled). The immobilized species is then washed to remove any unbound material and the bound material is detected. To prescreen large numbers of test agents, high throughput assays are generally preferred. Various assay formats are discussed in greater detail below.

B. Prescreening Based on Binding to CAP/SORBS1 Polynucleotides

The invention also provides a prescreening method based on screening test agents for specific binding to a CAP/SORBS1 polynucleotide. Agents that specifically bind to such polynucleotides have the potential to modulate the expression of the encoded polypeptide, and thereby modulate insulin/glucose homeostasis.

In one embodiment, therefore, a prescreening method of the invention entails contacting a test agent with a CAP/SORBS1 polynucleotide. The CAP/SORBS1 polynucleotide can be, for example, a CAP/SORBS1 gene, a CAP/SORBS1 promoter, and/or a CAP/SORBS1 coding sequence.

Specific binding of the test agent to the polynucleotide is determined. If specific binding is detected, the test agent is selected as potentially useful in the prophylaxis or treatment of a disorder of insulin homeostasis and/or glucose homeostasis.

Such prescreening is generally most conveniently accomplished with a simple in vitro binding assay, which are well known to those of skill in the art. In preferred binding assays, the polynucleotide is immobilized and exposed to a test agent (which can be labeled), or alternatively, the test agent(s) are immobilized and exposed to the polynucleotide (which can be labeled). The immobilized species is then washed to remove any unbound material and the bound material is detected. To prescreen large numbers of test agents, high throughput assays are generally preferred. Various assay formats are discussed in greater detail below.

C. Screening Based on Levels of CAP/SORBS1 or on Levels of CAP/SORBS1 Polynucleotides

Test agents, including, for example, those identified in a prescreening assay of the invention can also be screened to determine whether the test agent modulates CAPS/SORBS1 expression, e.g., by measuring the level(s) of CAP/SORBS1 polypeptides or polynucleotides (e.g., CAP/SORBS1 mRNA). Agents that modulate these levels can potentially modulate insulin/glucose homeostasis.

Accordingly, the invention provides a method of screening for an agent that modulates insulin/glucose homeostasis in which a test agent is contacted with a cell that expresses CAP/SORBS1 in the absence of test agent. Preferably, the method is carried out using an in vitro assay. In such assays, the test agent can be contacted with a cell in culture or present in a tissue. Alternatively, the test agent can be contacted with a cell lysate or fraction thereof. The level of CAP/SORBS1 polypeptides or polynucleotides (e.g., mRNA) is determined in the presence and absence (or presence of a lower amount) of test agent to identify any test agents that alter the level. If the level is altered, the test agent is selected as potentially useful in the prophylaxis or treatment of a disorder of insulin homeostasis and/or glucose homeostasis.

Cells or tissues useful in this screening method include those from any of the species described above in connection with knockout mammals. Cells that naturally express CAP/SORBS1 are typically, although not necessarily, employed in this screening method. Examples include, but are not limited to, 3T3L1 adipocytes and HepG2 hepatocytes. Alternatively, cells that have been engineered to express CAP/SORBS1 can be used in the method.

1. Sample

As noted above, screening assays are generally carried out in vitro, for example, in cultured cells, in a biological sample, or fractions thereof. For ease of description, cell cultures, biological samples, and fractions are referred to as “samples” below. The sample is generally derived from an animal, preferably from a mammal, and more preferably from a human.

The sample may be pretreated as necessary by dilution in an appropriate buffer solution or concentrated, if desired. Any of a number of standard aqueous buffer solutions, employing one or more of a variety of buffers, such as phosphate, Tris, or the like, at physiological pH can be used.

2. Polypeptide-Based Assays

CAP/SORBS1 can be detected and quantified by any of a number of methods well known to those of skill in the art. Examples of analytic biochemical methods suitable for detecting polypeptide include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immuno sorbent as says (ELISAs), immunofluorescent assays, Western blotting, and the like.

In one embodiment, CAP/SORBS1 is detected/quantified using a binding assay. Briefly, a sample from a tissue expressing the polypeptide of interest is incubated with a suitable binding partner (such as, e.g., an antibody) under conditions designed to provide a saturating concentration of the binding partner over the incubation period. After treatment with the binding partner, the sample is assayed for binding. Any binding partner that binds to the polypeptide of interest can be employed in the assay, although, if the polypeptide is one of a number of isoforms, binding partners specific for the particular isoform being assayed are preferred.

In another embodiment, CAP/SORBS1 are detected/quantified in an electrophoretic polypeptide separation (e.g. a 1- or 2-dimensional electrophoresis). Means of detecting polypeptides using electrophoretic techniques are well known to those of skill in the art (see generally, R. Scopes (1982) Polypeptide Purification, Springer-Verlag, N.Y.; Deutscher, (1990) Methods in Enzymology Vol. 182: Guide to Polypeptide Purification, Academic Press, Inc., N.Y.).

A variation of this embodiment utilizes a Western blot (immunoblot) analysis to detect and quantify the presence of CAP/SORBS1 in the sample. This technique generally comprises separating sample polypeptides by gel electrophoresis on the basis of molecular weight, transferring the separated polypeptides to a suitable solid support (such as a nitrocellulose filter, a nylon filter, or derivatized nylon filter), and incubating the support with antibodies that specifically bind the target polypeptide(s). Antibodies that specifically bind to the target polypeptide(s) may be directly labeled or alternatively may be detected subsequently using labeled antibodies (e.g., labeled sheep anti-mouse antibodies) that specifically bind to a domain of the primary antibody.

In a preferred embodiment, CAP/SORBS1 is detected and/or quantified in the biological sample using any of a number of well-known immunoassays (see, e.g., U.S. Pat. Nos. 4,366,241; 4,376,110; 4,517,288; and 4,837,168). For a general review of immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991).

Conventional immunoassays often utilize a “capture agent” to specifically bind to and often immobilize the analyte (in this case, CAP/SORBS1). In preferred embodiments, the capture agent is an antibody.

Immunoassays also typically utilize a “labeling agent” to specifically bind to and label the binding complex formed by the capture agent and the target polypeptide. The labeling agent may itself be one of the moieties making up the antibody/target polypeptide complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/target polypeptide complex. Other polypeptides capable of specifically binding immunoglobulin constant regions, such as polypeptide A or polypeptide G may also be used as the labeling agent. These polypeptides are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured target polypeptide is directly measured. In competitive assays, the amount of target polypeptide in the sample is measured indirectly by measuring the amount of an added (exogenous) polypeptide displaced (or competed away) from a capture agent by the target polypeptide present in the sample. In one competitive assay, a known amount of, in this case, labeled CAP/SORBS1 is added to the sample, and the sample is then contacted with a capture agent. The amount of labeled CAP/SORBS1 bound to the antibody is inversely proportional to the concentration of CAP/SORBS1 present in the sample.

Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads™), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horseradish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

In particular embodiments, immunoassays according to the invention are carried out using a MicroElectroMechanical System (MEMS). MEMS are microscopic structures integrated onto silicon that combine mechanical, optical, and fluidic elements with electronics, allowing convenient detection of an analyte of interest. An exemplary MEMS device suitable for use in the invention is the Protiveris' multicantilever array. This array is based on chemo-mechanical actuation of specially designed silicon microcantilevers and subsequent optical detection of the microcantilever deflections. When coated on one side with a protein, antibody, antigen, or DNA fragment, a microcantilever will bend when it is exposed to a solution containing the complementary molecule. This bending is caused by the change in the surface energy due to the binding event. Optical detection of the degree of bending (deflection) allows measurement of the amount of complementary molecule bound to the microcantilever.

Antibodies useful in these immunoassays include polyclonal and monoclonal antibodies.

3. Polynucleotide-Based Assays

Changes in CAP/SORBS1 expression level can be detected by measuring changes in levels of mRNA and/or a polynucleotide derived from the mRNA (e.g., reverse-transcribed cDNA, etc.).

Polynucleotides can be prepared from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

i. Amplification-Based Assays

In one embodiment, amplification-based assays can be used to detect, and optionally quantify, a polynucleotide encoding CAP/SORBS1. In exemplary amplification-based assays, CAP/SORBS1 mRNA in the sample acts as a template in an amplification reaction carried out with a nucleic acid primer that contains a detectable label or component of a labeling system. Suitable amplification methods include, but are not limited to, polymerase chain reaction (PCR); reverse-transcription PCR(RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of CAP/SORBS1 mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

ii. Hybridization-Based Assays

Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of CAP/SORBS1 mRNA can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of CAP/SORBS1 mRNA, and standard methods (such as, e.g., densitometry where the nucleic acid probe is radioactively labeled) can be used to quantify the level of CAP/SORBS1 mRNA).

A variety of additional nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to a solid support and a labeled “signal” nucleic acid in solution. The sample provides the target polynucleotide. The capture nucleic acid and signal nucleic acid each hybridize with the target polynucleotide to form a “sandwich” hybridization complex.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low-density” arrays can simply be produced by spotting (e.g., by hand using a pipette) different nucleic acids at different locations on a solid support (e.g., a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

In a particular embodiment, the arrays used in this invention contain “probe” nucleic acids. These probes are then hybridized respectively with their “target” nucleotide sequence(s) present in polynucleotides derived from a biological sample. Alternatively, the format can be reversed, such that polynucleotides from different samples are arrayed and this array is then probed with one or more probes, which can be differentially labeled.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, and/or enhance signal detection. If covalent bonding between a compound and the surface is desired, the surface will usually be polyfunctional or be capable of being polyfunctionalized. Functional groups that may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

Arrays can be made up of target elements of various sizes, ranging from about 1 mm diameter down to about 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

Hybridization assays according to the invention can also be carried out using a MicroElectroMechanical System (MEMS), such as the Protiveris' multicantilever array.

iii. Polynucleotide Detection

CAP/SORBS1 polynucleotides can be detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods described in the art are the nucleic acid sequence based amplification (NASBAO, Cangene, Mississauga, Ontario) and Q Beta Replicase systems.

In a preferred embodiment, suitable for use in amplification-based assays of the invention, a primer contains two fluorescent dyes, a “reporter dye” and a “quencher dye.” When intact, the primer produces very low levels of fluorescence because of the quencher dye effect. When the primer is cleaved or degraded (e.g., by exonuclease activity of a polymerase, see below), the reporter dye fluoresces and is detected by a suitable fluorescent detection system. Amplification by a number of techniques (PCR, RT-PCR, RCA, or other amplification method) is performed using a suitable DNA polymerase with both polymerase and exonuclease activity (e.g., Taq DNA polymerase). This polymerase synthesizes new DNA strands and, in the process, degrades the labeled primer, resulting in an increase in fluorescence. Commercially available fluorescent detection systems of this type include the ABI Prism® Systems 7000, 7700, or 7900 (TaqMan®) from Applied Biosystems or the LightCycler® System from Roche.

D. Screening Based on CAP/SORBS1 Activity

The invention also provides a screening method based on determining the effect, if any, of a test agent on the level of CAP/SORBS1 activity. CAP/SORBS1 activity can be assayed by measuring any activity of, or response mediated by, CAP/SORBS1. This can be achieved by determining the interactions among insulin receptor, CAP/SORBS1, Cbl, APS by standard protein-protein interaction experimental methods, and/or by measuring protein phosphorylation and protein kinase activities in the interacting proteins such as, but not limited to, Cbl, and TC10. Agents that modulate CAP/SORBS1 activity can potentially modulate insulin/glucose homeostasis.

Accordingly, the invention provides a method of screening for an agent that can modulate insulin/glucose homeostasis in which a test agent is contacted with a cell that expresses CAP/SORBS1 in the absence of test agent. Preferably, the method is carried out using an in vitro assay. When the test agent is contacted with a cell, the cell can be in culture or present in a tissue. The level of CAP/SORBS1 activity is determined in the presence and absence (or presence of a lower amount) of test agent to identify any test agents that alter the level. If the level of CAP/SORBS1 activity (attributable, for example, to the sequences affected in the knockout mice described herein) is reduced, the test agent is selected as potentially useful in the prophylaxis or treatment of a disorder of insulin homeostasis and/or glucose homeostasis.

Cells or tissues useful for screening based on CAP/SORBS1 activity include any of those described above in connection with screening based on levels of CAP/SORBS1 polypeptides or polynucleotides.

CAP/SORBS1 activity can be measured using any assay for an CAP/SORBS1 activity or CAP/SORBS 1-mediated response.

E. Test Agent Databases

In a preferred embodiment, generally involving the screening of a large number of test agents, the screening method includes the recordation of any test agent selected in any of the above-described prescreening or screening methods in a database of candidate modulators of insulin/glucose homeostasis.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

F. Test Agents Identified by Screening

When a test agent is found to alter CAP/SORBS1 expression or activity, a preferred screening method of the invention further includes combining the test agent with a carrier, preferably a pharmaceutically acceptable carrier, such as are described below in connection with treatment methods. Generally, the concentration of test agent is sufficient to alter the level of CAP/SORBS1 or its polynucleotide or activity(ies) when the composition is contacted with a cell. This concentration will vary, depending on the particular test agent and specific application for which the composition is intended. As one skilled in the art appreciates, the considerations affecting the formulation of a test agent with a carrier are generally the same as described below with respect to treatment methods.

In a preferred embodiment, the test agent is administered to an animal to measure the ability of the selected test agent to prevent or treat a disorder of insulin homeostasis and/or glucose homeostasis in a subject, as described in the next section.

G. Screening Based on Modulation of Insulin/Glucose Homeostasis In Vivo

The invention also provides an in vivo method of screening for an agent that that can modulate insulin/glucose homeostasis in a subject. In particular embodiments, the screening method is carried out to identify agents useful in the prophylaxis and/or treatment of a disorder such as metabolic syndrome, pre-diabetes, diabetes, and obesity.

In one embodiment, the method entails selecting an enhancer of the expression or activity of the CAP/SORBS1 sequences affected in the knockout mice described herein as a test agent, and measuring the ability of the selected test agent to prevent or treat a disorder of insulin homeostasis and/or glucose homeostasis in subject. Any agent that enhances CAP/SORBS1 and that can be administered to a subject can be employed in the method. Accordingly, test agents selected through any of the prescreening or screening methods of the invention can be tested in vivo. Alternatively, known enhancers of CAP/SORBS1 can be employed.

Test agents can be formulated for administration to a subject as described below in connection with treatment methods.

The subject of the method can be any individual that has CAP/SORBS1 and in which symptoms of insulin/glucose homeostasis can be measured. In certain embodiments, the subject is a non-human mammal. Examples of suitable subjects include research animals, such as mice, rats, guinea pigs, rabbits, cats, dogs, as well as monkeys and other primates, and humans. In preferred embodiments, an animal model established for studying a response to insulin/glucose homeostasis is employed.

Generally, an indicator(s) of CAP/SORBS1 expression or activity is measured and compared with that observed in the absence of test agent and/or in the presence of a lower amount of test agent. Test agents can be administered by any suitable route, as described below. The concentration of test agent is typically sufficient to alter the level of CAP/SORBS1 polypeptides, polynucleotides, or activity in vivo.

A preferred embodiment uses a mammal exhibiting a decreased level of CAP/SORBS1 as compared to a wild-type mammal. This embodiment entails administering a test agent to the mammal and determining an indicator of insulin/glucose homeostasis, such as glucose tolerance and/or insulin resistance. A test agent that modulates this indicator is selected as a candidate agent useful in the prophylaxis or treatment of a disorder of insulin/glucose homeostasis. In an exemplary variation of this embodiment, the mammal is a knockout mammal having a disruption in an endogenous CAP/SORBS1 gene, wherein the disruption results in the mammal exhibiting a decreased level of CAP/SORBS1 as compared to a wild-type mammal

### V. Diagnostic and Treatment Methods

A. Method Of Determining Whether a Subject is a Candidate for CAP/SORBS1-Based Therapy

The invention also provides a method of determining whether a subject is a candidate for CAP/SORBS1-based therapy. The method entails measuring the level and/or activity of CAP/SORBS1 in a biological sample from the subject. An abnormal level or activity (e.g., a reduced level or activity, relative to a normal level) of CAP/SORBS1 indicates that the subject is a candidate for CAP/SORBS1-based therapy.

The subject can be from any species that expresses CAP/SORBS1, including any of those described herein. Typically, the subject will be human or a species having commercial value or value as a pet. In particular embodiments, the subject has at least one symptom of a disorder of insulin/glucose homeostasis. Examples of such disorders include, but are not limited to, metabolic syndrome, pre-diabetes, diabetes, and obesity. Examples of such symptoms include: hyperinsulinemia, hyperlipidemia, hypertension, artherosclerosis, vascular diseases, polycystic ovarian syndrome, fatty liver, acanthosis nigricans, gouty arthritis, and metabolic syndrome-related inflammation.

Suitable biological samples include any sample expected to contain measurable CAP/SORBS1 (e.g., adipose tissue, muscle, liver, blood cells, brain, heart, blood vessels, kidney, etc). Levels and activities of CAP/SORBS1 can be measured using any suitable assay, such as for example any of those described above. The invention encompasses measuring indirect indicators of CAP/SORBS1 levels, such as CAP/SORBS1 mRNA levels. As those of skill in the art readily appreciate, a reduction in CAP/SORBS1 mRNA levels will typically correlate with a reduction in CAP/SORBS1 protein and, consequently, activity, levels.

A CAP/SORBS1 level outside of a normal range indicates that the subject is a candidate for a therapy aimed at bringing the CAP/SORBS1 into the normal range. A reduced or increased CAP/SORBS1 level can be indicative of a disorder of insulin/glucose homeostasis, such as metabolic syndrome, pre-diabetes, diabetes, and obesity. Thus, the determination that CAP/SORBS1 is reduced in a subject, e.g., one suspected or known to be at risk for, or have, such a disorder, indicates that the enhancement of CAP/SORBS1 expression and/or activity is likely to ameliorate one or more symptoms of the disorder. In certain embodiments, the method of the invention additionally entails treating such a subject with an enhancer of CAP/SORBS1, as described in greater detail below.

Any agent that increases CAP/SORBS1 activity, if it is low, or that decreases CAP/SORBS1 activity, if it is high, can be administered, including CAP/SORBS1 polypeptides and/or polynucleotides. Agonists of peroxisome proliferator activated receptor gamma (PPARγ) have been shown to enhance CAP/SORBS1 expression and can thus be employed in the method of the invention. PPARγ agonists and their use in the treatment of diabetes are known and described, for example, in U.S. Pat. Nos. 7,186,746; 7,179,830; 7,157,581; 7,153,878; and 7,119,221.

B. Method of Prophylaxis or Treatment of a Disorder of Insulin/Glucose Homeostasis

In particular embodiments, the invention provides a method of prophylaxis or treatment of a disorder of insulin homeostasis and/or glucose homeostasis. The method entails administering an effective amount of an agent that modulates CAP/SORBS1 activity to a subject at risk for, or having, a disorder of insulin homeostasis and/or glucose homeostasis, provided that the level of CAP/SORBS1 activity is modulated by means other than treatment with an agonist of peroxisome proliferator activated receptor gamma (PPARγ).

The subject can be from any species that expresses CAP/SORBS1, including any of those described herein. Typically, the subject will be human or a species having commercial value or value as a pet. Suitable subjects include those identified as at risk for, or having, a disorder of insulin/glucose homeostasis. In particular embodiments, the subject has at least one symptom of a disorder of insulin/glucose homeostasis. Examples of such disorders include, but are not limited to, metabolic syndrome, pre-diabetes, diabetes, and obesity. Accordingly, suitable subjects include those diagnosed as at risk for, or having, and of these disorders and/or having one or more of the following symptoms: hyperinsulinemia, hyperlipidemia, hypertension, artherosclerosis, vascular diseases, polycystic ovarian syndrome, fatty liver, acanthosis nigricans, gouty arthritis, and metabolic syndrome-related inflammation.

Any agent or combination of agents that enhances or reduces CAP/SORBS1 activity, as appropriate, can be employed, provided that the agent is not a PPARγ agonist by itself. Suitable agents include CAP/SORBS1 polypeptides and polynucleotides, as well as any agent identified in a screening method herein. Exemplary CAP/SORBS1 polypeptides include an amino acid sequence that has at least about 70% identity to a wild-type CAP/SORBS1 amino acid sequence over a comparison window of at least 15 contiguous amino acids. Percent identity can, for example, be determined by a sequence alignment performed using BLASTP with default parameters set to measure 70% identity. In variations of this embodiment, the percent identity is 80, 90, 95, 96, 97, 98, 99, or 100 percent.

A CAP/SORBS1 polypeptide can be administered directly or indirectly, e.g., by administering a composition containing a polynucleotide encoding CAP/SORBS1 polypeptide to the subject. In the latter embodiment, this administration results in the introduction of the polynucleotide into one or more cells and the subsequent expression of the polypeptide in an amount sufficient to alter CAP/SORBS1 activity.

In particular embodiments, the treatment method additionally entails determining glucose tolerance, and/or insulin resistance, and/or metabolic syndrome in the subject after treatment to alter CAP/SORBS1 activity to determine whether the subject is responding to the treatment. Glucose tolerance and insulin resistance can be determined using any suitable test, such as those described below in the examples. Determination of metabolic syndrome entails identification of at least three of the following: hyperglycemia; hyperlipidemia; hypertension; and obesity. Other disorders related to the metabolic syndrome include hyperinsulinemia, artherosclerosis, vascular diseases, polycystic ovarian syndrome, fatty liver, acanthosis nigricans, gouty arthritis, and inflammation.

C. Compositions

For research and therapeutic applications, a CAP/SORBS1 modulator is generally formulated to deliver modulator to a target site in an amount sufficient to modulate the CAP/SORBS1 at that site (e.g., adipose tissue and/or muscle). Modulator compositions according to the invention optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980.

A pharmaceutically acceptable carrier suitable for use in the invention is non-toxic to cells, tissues, or subjects at the dosages employed, and can include a buffer (such as a phosphate buffer, citrate buffer, and buffers made from other organic acids), an antioxidant (e.g., ascorbic acid), a low-molecular weight (less than about 10 residues) peptide, a polypeptide (such as serum albumin, gelatin, and an immunoglobulin), a hydrophilic polymer (such as polyvinylpyrrolidone), an amino acid (such as glycine, glutamine, asparagine, arginine, and/or lysine), a monosaccharide, a disaccharide, and/or other carbohydrates (including glucose, mannose, and dextrins), a chelating agent (e.g., ethylenediaminetetratacetic acid [EDTA]), a sugar alcohol (such as mannitol and sorbitol), a salt-forming counterion (e.g., sodium), and/or an anionic surfactant (such as Tween™, Pluronics™, and PEG). In one embodiment, the pharmaceutically acceptable carrier is an aqueous pH-buffered solution.

Particular embodiments include sustained-release pharmaceutical compositions. An exemplary sustained-release composition has a semipermeable matrix of a solid hydrophobic polymer to which the modulator is attached or in which the modulator is encapsulated. Examples of suitable polymers include a polyester, a hydrogel, a polylactide, a copolymer of L-glutamic acid and T-ethyl-L-glutamase, non-degradable ethylene-vinylacetate, a degradable lactic acid-glycolic acid copolymer, and poly-D-(−)-3-hydroxybutyric acid. Such matrices are typically in the form of shaped articles, such as films, or microcapsules.

Where the modulator is a polypeptide, exemplary sustained release compositions include the polypeptide attached, typically via ε-amino groups, to a polyalkylene glycol (e.g., polyethylene glycol [PEG]). Attachment of PEG to proteins is a well-known means of reducing immunogenicity and extending in vivo half-life (see, e.g., Abuchowski, J., et al. (1977) J. Biol. Chem. 252:3582-86. Any conventional “pegylation” method can be employed, provided the “pegylated” protein retains the desired function(s).

In another embodiment, a sustained-release composition includes a liposomally entrapped modulator. Liposomes are small vesicles composed of various types of lipids, phospholipids, and/or surfactants. These components are typically arranged in a bilayer formation, similar to the lipid arrangement of biological membranes. Liposomes containing modulators according to the invention are prepared by known methods, such as, for example, those described in Epstein, et al. (1985) PNAS USA 82:3688-92, and Hwang, et al., (1980) PNAS USA, 77:4030-34. Ordinarily the liposomes in such preparations are of the small (about 200-800 Angstroms) unilamellar type in which the lipid content is greater than about 30 mol. percent cholesterol, the specific percentage being adjusted to provide the optimal therapy. Useful liposomes can be generated by the reverse-phase evaporation method, using a lipid composition including, for example, phosphatidylcholine, cholesterol, and PEG-derivatized phosphatidylethanolamine (PEG-PE). If desired, liposomes are extruded through filters of defined pore size to yield liposomes of a particular diameter.

Pharmaceutical compositions of the invention can be stored in any standard form, including, e.g., an aqueous solution or a lyophilized cake. Such compositions are typically sterile when administered to subjects. Sterilization of an aqueous solution is readily accomplished by filtration through a sterile filtration membrane. If the composition is stored in lyophilized form, the composition can be filtered before or after lyophilization and reconstitution.

In particular embodiments, the methods of the invention employ pharmaceutical compositions containing a polynucleotide modulator or a polynucleotide encoding a polypeptide modulator of CAP/SORBS1. Such compositions optionally include other components, as for example, a storage solution, such as a suitable buffer, e.g., a physiological buffer. In a preferred embodiment, the composition is a pharmaceutical composition and the other component is a pharmaceutically acceptable carrier, as described above.

Preferably, compositions containing polynucleotides useful in the invention also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with polynucleotides useful in the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC).

In another embodiment, polynucleotides are complexed to dendrimers, which can be used to introduce polynucleotides into cells. Dendrimer polycations are three-dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

For therapeutic use, polynucleotides useful in the invention are formulated in a manner appropriate for the particular indication. U.S. Pat. No. 6,001,651 to Bennett et al. describes a number of pharmaceutical compositions and formulations suitable for use with an oligonucleotide therapeutic as well as methods of administering such oligonucleotides.

D. Administration

Pharmaceutical compositions according to the invention are generally administered according to known methods for administering small-molecule drugs, as well as therapeutic polypeptides, peptides, and polynucleotides them. Suitable routes of administration include, for example, topical, intravenous, intraperitoneal, intracerebral, intraventricular, intramuscular, intraocular, intraarterial, or intralesional routes. Pharmaceutical compositions of the invention can be administered continuously by infusion, by bolus injection, or, where the compositions are sustained-release preparations, by methods appropriate for the particular preparation.

In certain embodiments, the compositions are delivered through the skin using a conventional transdermal drug delivery system, i.e., a transdermal “patch” wherein the composition is typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of a selected composition that is ultimately available for delivery to the surface of the skin. Thus, for example, the reservoir may include the composition in an adhesive on a backing layer of the patch, or in any of a variety of different matrix formulations known to those of skill in the art. The patch may contain a single reservoir, or it may contain multiple reservoirs.

In other embodiments, the compositions of the invention are administered in implantable depot formulations. A wide variety of approaches to designing depot formulations that provide sustained release of an active agent are known and are suitable for use in the invention. Generally, the components of such formulations are biocompatible and may be biodegradable. Biocompatible polymeric materials have been used extensively in therapeutic drug delivery and medical implant applications to effect a localized and sustained release. See Leong et al., “Polymeric Controlled Drug Delivery”, Advanced Drug Delivery Rev., 1:199-233 (1987); Langer, “New Methods of Drug Delivery”, Science, 249:1527-33 (1990); Chien et al., Novel Drug Delivery Systems (1982). Such delivery systems offer the potential of enhanced therapeutic efficacy and reduced overall toxicity.

If an implant is intended for use as a drug delivery or other controlled-release system, using a biodegradable polymeric carrier is one effective means to deliver the therapeutic agent locally and in a controlled fashion, see Langer et al., “Chemical and Physical Structures of Polymers as Carriers for Controlled Release of Bioactive Agents”, J. Macro. Science, Rev. Macro. Chem. Phys., C23(1), 61-126 (1983). As a result, less total drug is required, and toxic side effects can be minimized. Examples of classes of synthetic polymers that have been studied as possible solid biodegradable materials include polyesters (Pitt et al., “Biodegradable Drug Delivery Systems Based on Aliphatic Polyesters: Applications to Contraceptives and Narcotic Antagonists,” Controlled Release of Bioactive Materials, 19-44 (Richard Baker ed., 1980); poly(amino acids) and pseudo-poly(amino acids) (Pulapura et al. “Trends in the Development of Bioresorbable Polymers for Medical Applications,” J. Biomaterials Appl., 6:1, 216-50 (1992); polyurethanes (Bruin et al., “Biodegradable Lysine Diisocyanate-based Poly(Glycolide-co-.epsilon. Caprolactone)-Urethane Network in Artificial Skin,” Biomaterials, 11:4, 291-95 (1990); polyorthoesters (Heller et al., “Release of Norethindrone from Poly(Ortho Esters),” Polymer Engineering Sci., 21:11, 727-31 (1981); and polyanhydrides (Leong et al., “Polyanhydrides for Controlled Release of Bioactive Agents,” Biomaterials 7:5, 364-71 (1986).

Thus, for example, a CAP/SORBS1 modulator composition can be incorporated into a biocompatible polymeric composition and formed into the desired shape outside the body. This solid implant is then typically inserted into the body of the subject through an incision. Alternatively, small discrete particles composed of these polymeric compositions can be injected into the body, e.g., using a syringe. In an exemplary embodiment, an modulator composition can be encapsulated in microspheres of poly (D,L-lactide) polymer suspended in a diluent of water, mannitol, carboxymethyl-cellulose, and polysorbate 80. The polylactide polymer is gradually metabolized to carbon dioxide and water, releasing the modulater into the system.

In yet another approach, depot formulations can be injected via syringe as a liquid polymeric composition. Liquid polymeric compositions useful for biodegradable controlled release drug delivery systems are described, e.g., in U.S. Pat. Nos. 4,938,763; 5,702,716; 5,744,153; 5,990,194; and 5,324,519. After injection in a liquid state or, alternatively, as a solution, the composition coagulates into a solid.

One type of polymeric composition suitable for this application includes a nonreactive thermoplastic polymer or copolymer dissolved in a body fluid-dispersible solvent. This polymeric solution is placed into the body where the polymer congeals or precipitates and solidifies upon the dissipation or diffusion of the solvent into the surrounding body tissues. See, e.g., Dunn et al., U.S. Pat. Nos. 5,278,201; 5,278,202; and 5,340,849 (disclosing a thermoplastic drug delivery system in which a solid, linear-chain, biodegradable polymer or copolymer is dissolved in a solvent to form a liquid solution).

A CAP/SORBS1 modulator composition can also be adsorbed onto a membrane, such as a silastic membrane, which can be implanted, as described in International Publication No. WO 91/04014.

E. Dose

The dose of modulator is sufficient to enhance or alter CAP/SORBS1 activity, preferably without significant toxicity. In particular in vivo embodiments, the amount of the modulator is sufficient to improve one or more measures of insulin/glucose homeostasis in a subject.

The dose of modulator depends, for example, upon the therapeutic objectives, the route of administration, and the condition of the subject. Accordingly, it is necessary for the clinician to titer the dosage and modify the route of administration as required to obtain the optimal therapeutic effect. Generally, the clinician begins with a low dose and increases the dosage until the desired therapeutic effect is achieved. Starting doses for a given modulator can be extrapolated from in vitro data for a the modulater.

### VI. Kits

The invention also provides kits useful in practicing the methods of the invention. In one embodiment, a kit of the invention includes one or more reagents useful for detecting a CAP/SORBS1 polypeptide or polynucleotide in one or more suitable containers. In another embodiment, a kit of the invention includes a CAP/SORBS1 modulator in a suitable container. In a variation of this embodiment, the modulator is formulated in a pharmaceutically acceptable carrier. The kit preferably includes instructions for administering the modulator to a subject to improve insulin/glucose homeostasis.

Instructions included in kits of the invention can be affixed to packaging material or can be included as a package insert. While the instructions are typically written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

### EXAMPLES

The following examples are offered to illustrate, but not to limit, the claimed invention.

### Example 1

### CAP/SORBS1 Knockout Mice are Insulin Resistant and Glucose Intolerant

### Abstract

The molecular defects underlying the development of most human Type 2 DM are not yet clear. But insulin resistance plays a critical role during the development of Type 2 DM. Insulin actions are mediated through insulin receptor. Recent transgenic studies have shown that defects in insulin receptor signaling may lead to insulin resistance and glucose intolerance in experimental animals. Insulin receptor triggers complex intracellular signaling network; three major pathways have been implicated as potentially important in the regulation of glucose homeostasis, the IRS/PI3 kinase/Akt pathway, the CAP/APS/Cbl/TC10 pathway, and the phospholipase C/IP3 pathway. The importance of IRS/PI3 kinase pathway in the regulation of glucose homeostasis is well established. But whether insulin regulation of glucose homeostasis in vivo involves activation of CAP/TC10 pathway remained an unanswered question because in vitro studies in cultured cells had yielded conflicting results. To investigate the role of CAP in insulin resistance and glucose tolerance, homozygous CAP^{(-/-)} knockout mice were produced. CAP^{(-/-)} mice displayed higher blood glucose levels during glucose tolerance test. Without being bound by any theory, the data indicate that impairment of insulin receptor signaling mediated by CAP leads to insulin resistance and glucose intolerance in vivo. This new knockout model will be a useful tool to define the specific mechanisms through which CAP modulates glucose homeostasis.

### Generation of CAP Knockout Mice

To generate CAP knockout mice, three clones of ES cells were obtained from BayGenomics and the Mutant Mouse Regional Resource Center at UC Davis. These three cell lines (RRD158, RRP094, and RRJ558) contain insertional mutations in the CAP/SORBS1/SH3P12/Ponsin gene, which were created by gene trapping strategy. CAP has been mapped to chromosome 19qC3, and the locations and sequences of these insertions had been independently confirmed by BayGenomics and our laboratory. The gene trapping vector pGTlLxf was designed to create an in-frame fusion between the 5′ exons of the trapped gene and a reporter P3-Geo (fusion of β-galactosidase and neomycin phosphotransferase II). The ES cell clones were microinjected into blastocysts to produce chimera mice and then generate heterozygous CAP(+/−) mice. Knockout mice were obtained from two different ES clones, RRD158 and RRP094. The RRJ558 clone did not generate germline transmission. The gene trapped locus is between exon 4 and 5 in the RRD158 clone and between exon 17 and 18 in the RRP094 clone. Two different lines of CAP knockout mice, D158a and P094a, were produced on C57BL/6 and 129/Ola mixed genetic background. Homozygous RRD158a and RRD158b CAP(−/−) mice were live born in Mendelian ratio. The mice were housed in a controlled transgenic facility with 12-hour dark/light cycle and fed with standard chow. D158a and PO₉₄a have been respectively backcrossed to C57BL/6 for four generations.

### Genotyping of CAP Knockout Mice

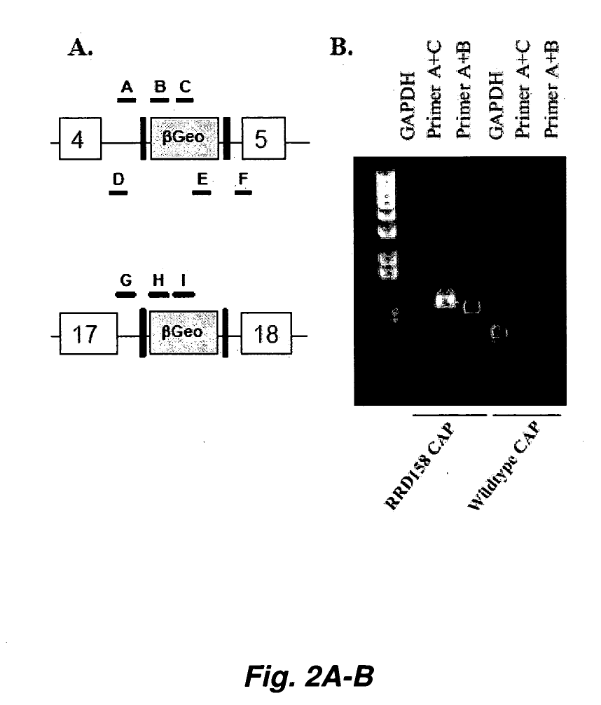

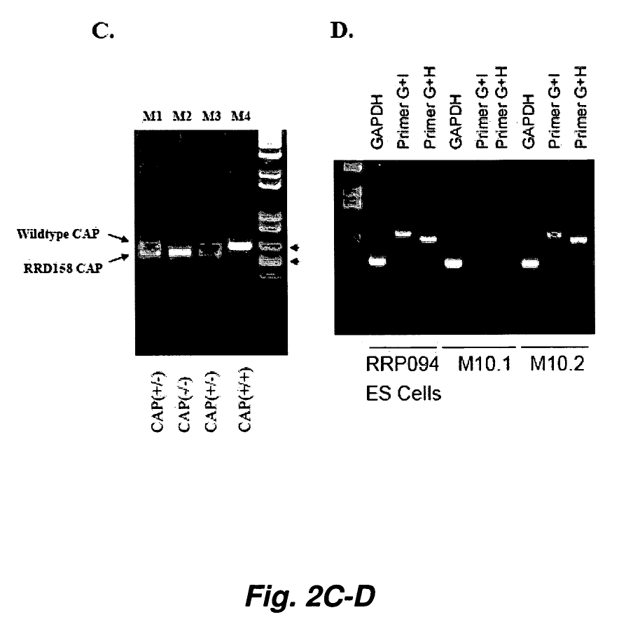

Genomic DNA from tail biopsy was used for genotyping of transgenic mice (FIG. 2). The PCR primers used for RRD158 and RRP094 genotyping are listed in FIG. 2, upstream primer A (5′-GCCCCTACCACTTTACCCTTTC-3′; (SEQ ID NO:1)) corresponds to exon 4 sequences near its 3′ end, downstream primer B (5′-ATTCAGGCTGCGCAACTGTTGGG-3′; (SEQ ID NO:2)) and downstream primer C (5′-AGTATCGGCCTCAGGAAGATCG-3′; (SEQ ID NO:3)) corresponds to β-Geo sequences. For confirmation of insertional mutation and to differentiate homozygous from heterozygous KO mice, the following primer sets were used for PCR. Upstream primer D (5′-ACACAGTTCCTCGACTTGCC-3′; (SEQ ID NO:4)) corresponds to part of the intron sequences between exons 4 and 5 and downstream primer F (5′-CGAAAAGTGCCACCTGACGTC-3′; (SEQ ID NO:5)) corresponds to 3′ end of the intron and the 5′ end of exon 5. The sequence of these PCR products have all been sequenced and confirmed. Using similar strategy, we also identified CAP knockout mice derived from RRP094 ES clone (FIG. 2D).

### Expression of CAP in CAP^{(-/-)} Tissues

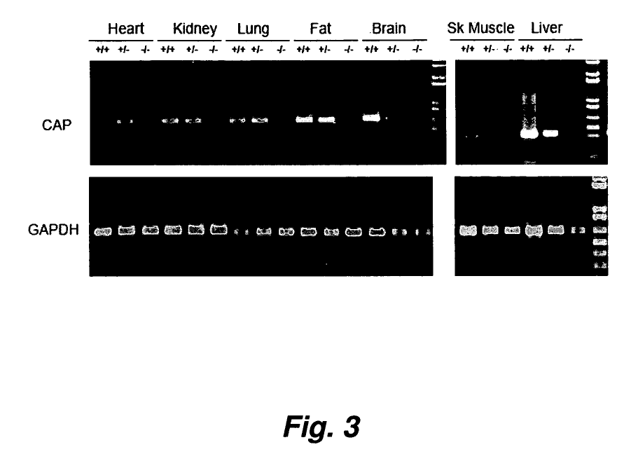

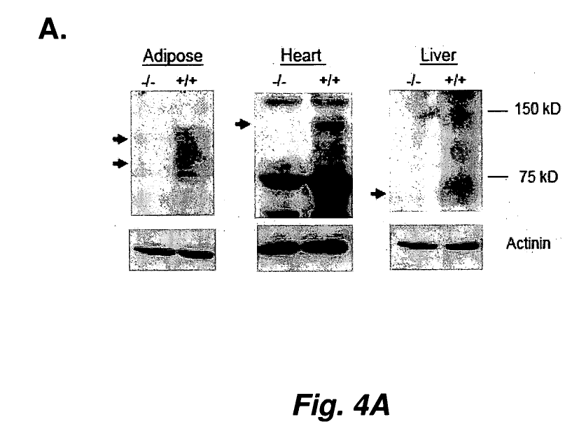

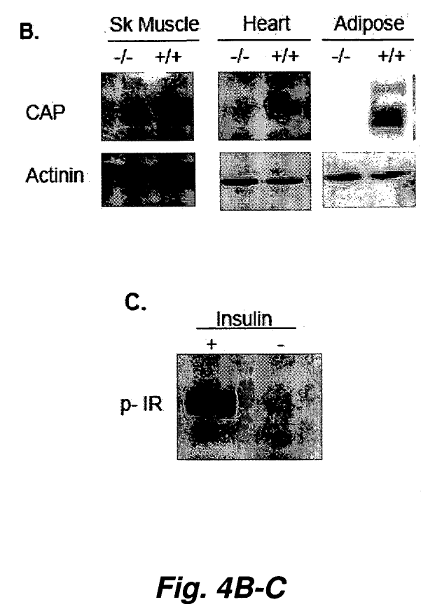

The heterozygous and homozygous CAP KO mice appear to be grossly normal. As shown in the FIG. 3, wildtype CAP mRNA could not be detected in the heart, kidney, lung, brain, skeletal muscle, and liver in the homozygous knockout mice. Two alternatively-spliced CAP isoforms were detected in the wildtype skeletal muscle, using the PCR primer set flanking exon 2 to exon 6. These DNA fragments (RT-PCR products) had been subcloned and sequenced; the results confirmed that these fragments correspond to the expected sequence of CAP gene. In addition to mRNA levels, the content of CAP protein was determined in these mice. Proteins were extracted from CAP^{(+/+)} and CAP^{(-/-)} mice and resolved with 8% SDS-PAGE and immunoblotted with antibodies against CAP/SORBS1 (FIG. 4). Tissue-specific CAP isoforms expressed as proteins of various sizes in different tissues (60-150 kD). The dominant form of CAP proteins in fat and liver were nearly undetectable in the CAP^{(-/-)} mice. The photo of anti-CAP western blot in wildtype adipose tissue is similar to the photo of adipocytes CAP western blot posted on the antibody manufacturer's website (www.upstate.com). A 135 kD and a 62 kD CAP were knocked out in the heart, however, CAP antibodies pick up many non-specific bands in myocardium because prolonged exposure was needed to visualize the CAP band. These data indicate that CAP has been disrupted in various insulin-sensitive tissues in D158a and P094a CAP^{(-/-)} mice.

To study insulin receptor signaling in mice, the CAP^{(-/-)} mice were anesthetized and injected with insulin via inferior vena cava, and tyrosine phosphorylation of insulin receptor beta subunits was measured, as shown in FIG. 4B. This experiment established the feasibility of studying insulin receptor signaling in vivo in mice.

### Disruption of CAP Lead to Glucose Intolerance and Insulin Resistance

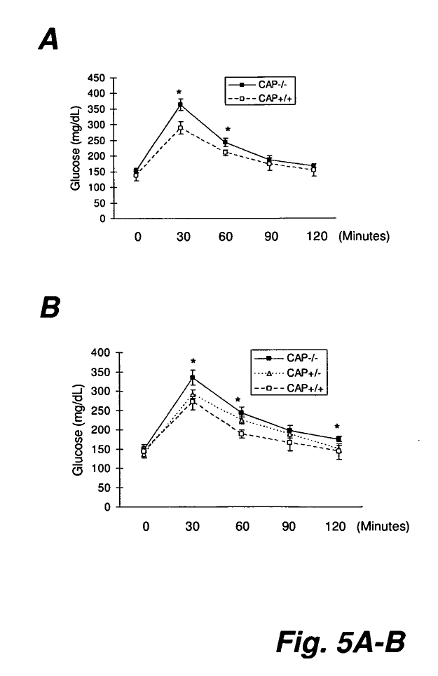

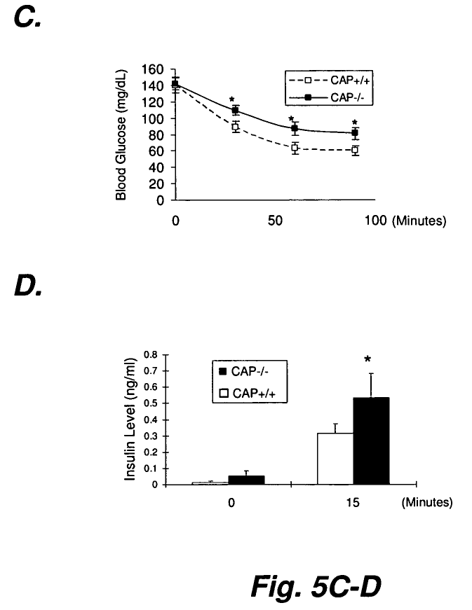

The mice are grossly normal with no visible difference in their appearance. After 5-hour fasting, glucose tolerance test (GTT) was carried out and the results were shown in FIG. 5. Postprandial glucose levels were significantly elevated in the female and male CAP(−/−) mice, suggesting glucose intolerance upon disruption of CAP (Female: FIG. 5A; Male: FIG. 5B). Insulin tolerance test showed lower insulin sensitivity in the female CAP KO mice (FIG. 5C). To determine insulin response upon glucose injection, plasma insulin levels were measured before and after glucose injection in the female mice (FIG. 5D). Plasma insulin levels were higher in the female homozygous CAP(−/−) mice, which again suggested the presence of insulin resistance.

### Disruption of CAP Did Not Change Body Weight

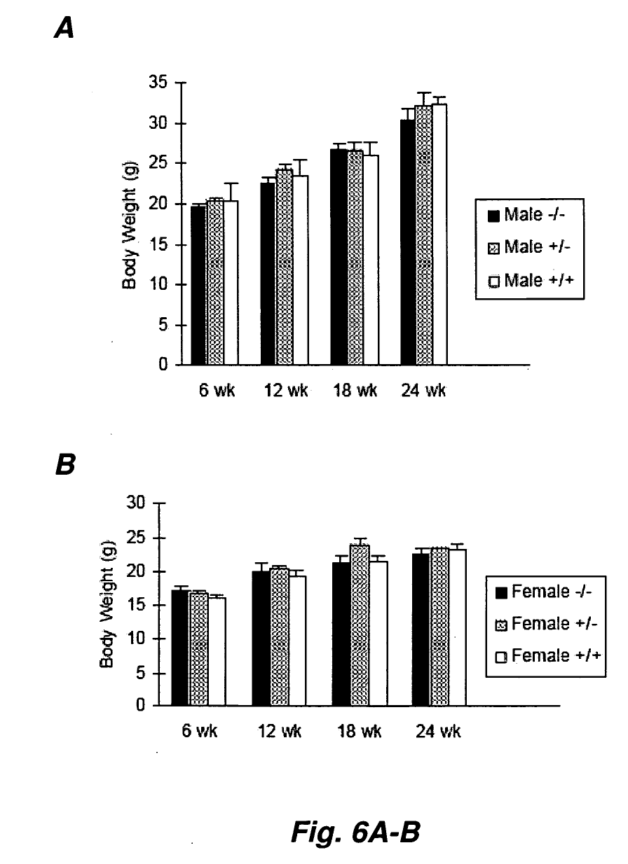

Body weight and epididymal fat weight were not different in wildtype littermate control and homozygous CAP^{(-/-)} mice (up to 24 weeks) in the D158a line (FIG. 6 and FIG. 7). Food intake was not altered in the knockout mice (FIG. 7). Therefore CAP did not play a role in the regulation of body weight or food intake when the mice were fed with regular diet, which suggests that the glucose intolerance in CAP^{(-/-)} mice was not secondary to obesity.

### CONCLUSION

The data showed that CAP^{(-/-)} mice were associated with glucose intolerance, insulin resistance, and hyperinsulinemia. These results indicate that CAP is involved in the regulation of glucose homeostasis mediated by insulin receptor. Body weight and food intake were not different between the CAP^{(-/-)} male mice and wildtype littermate controls. This is different from the Cbl knockout phenotype (increased insulin sensitivity, reduced adiposity, increased energy expenditure, increased food intake), which essentially ruled out the possibility that CAP suppressed inhibition of AMPK by Cbl and thus reduced energy expenditure, increased adiposity, and reduced insulin sensitivity in the knockout mice.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A non-human knockout mammal, the mammal comprising a disruption in an endogenous CAP/SORBS1 gene, wherein the disruption results in the mammal exhibiting a decreased level of CAP/SORBS1 as compared to a wild-type mammal.

2. The mammal of claim 1, wherein the mammal is selected from the group consisting of a rodent, an equine, a bovine, a porcine, a lagomorph, a feline, a canine, a murine, a caprine, an ovine, and a non-human primate.

3. The mammal of claim 1, wherein the mammal is a mouse.

4. The mammal of claim 1, wherein the disruption is selected from the group consisting of an insertion, a deletion, a frameshift mutation, a substitution, and a stop codon.

5. The mammal of claim 4, wherein the disruption comprises an insertion of an expression cassette into the endogenous CAP/SORBS1 gene.

6. The mammal of claim 5, wherein said expression cassette comprises a selectable marker.

7. The mammal of claim 5, wherein the expression cassette comprises a fusion of β-galactosidase and neomycin phosphotransferase II.

8. The mammal of claim 1, wherein said disruption is in a somatic cell.

9. The mammal of claim 1, wherein said disruption is in a germ cell.

10. The mammal of claim 1, wherein the mammal is homozygous for the disrupted CAP/SORBS gene.

11. The mammal of claim 1, wherein the mammal is heterozygous for the disrupted CAP/SORBS gene.

12. A method of prescreening for an agent useful in the prophylaxis or treatment of a disorder of insulin homeostasis and/or glucose homeostasis, the method comprising:
(a) contacting a test agent with a CAP/SORBS1 polypeptide;
(b) determining whether the test agent specifically binds to the polypeptide; and
(c) if the test agent specifically binds to the polypeptide, selecting the test agent as potentially useful in the prophylaxis or treatment of a disorder of insulin homeostasis and/or glucose homeostasis.

13. A method of prescreening for an agent useful in the prophylaxis or treatment of a disorder of insulin homeostasis and/or glucose homeostasis, the method comprising:
(a) contacting a test agent with a CAPS/SORBS1 polynucleotide;
(b) determining whether the test agent specifically binds to the polynucleotide; and
(c) if the test agent specifically binds to the polynucleotide, selecting the test agent as potentially useful in the prophylaxis or treatment of a disorder of insulin homeostasis and/or glucose homeostasis.

14. (canceled)

15. A method of screening for an agent useful in the prophylaxis or treatment of a disorder of insulin homeostasis and/or glucose homeostasis, the method comprising:
(a) contacting a test agent with a cell that expresses CAP/SORBS1 in the absence of test agent, or with a fraction of said cell;
(b) determining whether the test agent modulates CAP/SORBS1 expression and/or activity; and
(c) if the test agent modulates CAP/SORBS1 expression and/or activity, selecting the test agent as potentially useful in the prophylaxis or treatment of a disorder of insulin homeostasis and/or glucose homeostasis.

16-19. (canceled)

20. A method of screening for an agent useful in the prophylaxis or treatment of a disorder of insulin homeostasis and/or glucose homeostasis, the method comprising:
(a) selecting a modulator of CAP/SORBS1 expression or activity as a test agent; and
(b) measuring the ability of the selected test agent to prevent or treat a disorder of insulin homeostasis and/or glucose homeostasis in an animal model.

21. (canceled)

22. A method of screening for an agent useful in the prophylaxis or treatment of a disorder of insulin homeostasis and/or glucose homeostasis, the method comprising:
administering a test agent to a mammal exhibiting an abnormal level of CAP/SORBS1; and
determining one or more parameters selected from the group consisting of glucose tolerance, insulin resistance, and insulin sensitivity;
wherein a test agent that modulates said one or more parameters is selected as a candidate agent useful in the prophylaxis or treatment of a disorder of insulin homeostasis and/or glucose homeostasis.

23-26. (canceled)

27. A method of determining whether a subject is a candidate for CAP/SORBS1-based therapy, the method comprising:
measuring the level and/or activity of CAP/SORBS1 in a biological sample from the subject;
wherein an altered level or activity of CAP/SORBS1, relative to a normal level, indicates that the subject is a candidate for CAP/SORBS1-based therapy.

28-31. (canceled)

32. A method of prophylaxis or treatment of a disorder of insulin homeostasis and/or glucose homeostasis, the method comprising administering an agent that modulates CAP/SORBS1 activity to a subject at risk for, or having, a disorder of insulin homeostasis and/or glucose homeostasis, provided that said level of CAP/SORBS1 activity is increased by means other than treatment with an agonist of peroxisome proliferator activated receptor gamma (PPARγ).

33-36. (canceled)


SEQUENCE LISTING (5 sequences)

SEQ ID NO: 1 - DNA, 22 bp, Mus musculus
gcccctaccactttaccctttc

SEQ ID NO: 2 - DNA, 23 bp, Artificial
  Fusion of beta-galactosidase and neomycin phosphotransferase II
attcaggctgcgcaactgttggg

SEQ ID NO: 3 - DNA, 22 bp, Artificial
  Fusion of beta-galactosidase and neomycin phosphotransferase II
agtatcggcctcaggaagatcg

SEQ ID NO: 4 - DNA, 20 bp, Mus musculus
acacagttcctcgacttgcc

SEQ ID NO: 5 - DNA, 21 bp, Mus musculus
cgaaaagtgccacctgacgtc